(19) United States
(12) Patent Application Publication
(10) Pub. No.: US 2004/0038888 A1
(43) Pub. Date: Feb. 26, 2004

(54) Functional role and potential therapeutic use of PACAP, VIP and Maxadilan in relation to adult neural stem or progenitor cells

(76) Inventors: Alex Mercer, Bromma (SE); Cesare Patrone, Hagersten (SE); Harriet Ronnholm, Trangsund (SE); Lilian Wikstrom, Spanga (SE)

Correspondence Address: Ivor R. Elrifi, Mintz, Levin, Cohn, Ferris,, Glovsky and Popeo, P.C., 666 Third Avenue, 24th Floor, New York, NY, 10017, US

(21) Appl. No.: 10/429,062

(22) Filed: May 2, 2003

Related U.S. Application Data
(60) Provisional application No. 60/377,734, filed on May 3, 2002.
(60) Provisional application No. 60/393,264, filed on Jul. 2, 2002.
(60) Provisional application No. 60/426,827, filed on Nov. 15, 2002.

(51) Int. Cl. A61K 38/17

(52) U.S. Cl. 514/12

(57) ABSTRACT

The invention relates generally to methods of influencing central nervous system cells to produce progeny useful in the treatment of CNS disorders. More specifically, the invention includes methods of exposing a patient suffering from such a disorder to a reagent that modulates the proliferation, migration, differentiation and survival of central nervous system cells via PACAP, Maxadilan or VIP signaling. These methods are useful for reducing at least one symptom of the disorder.

### RELATED APPLICATIONS

This application claims the benefit of U.S. Ser. No. 60/377,734 filed May 3, 2002; U.S. Ser. No. 60/393,264 filed Jul. 2, 2002; and U.S. Ser. No. 60/426,827 filed Nov. 15, 2002. The contents of these applications are incorporated herein by reference in their entirety.

### FIELD OF THE INVENTION

The invention relates generally to methods of influencing adult neural stem cells and neural progenitor cells to produce progeny that can replace damaged or missing neurons or other central nervous system (CNS) cell types. More specifically, the invention includes methods of exposing a patient suffering from a disorder to a reagent that regulates the differentiation, proliferation, survival and migration of central nervous system cells via modulation of pituitary adenylate cyclase-activating polypeptide (PACAP), Maxadilan or VIP signalling. These methods are useful for reducing at least one symptom of a neurological disorder.

### BACKGROUND OF THE INVENTION

Throughout this specification, various patents, published patent applications and scientific references are cited to describe the state and content of the art. Those disclosures, in their entireties, are hereby incorporated into the present specification by reference.

For several years, it has been known that neural stem cells exist in the adult mammalian brain. This concept is of particular importance since the adult brain was thought to have very limited regenerative capacity. Moreover, the possibility to use adult-derived stem cells for tissue repair may help to overcome the ethical problems associated with embryonic cell research. Although the generation of neurons and glia can be observed in the adult brain, there is thus far only limited knowledge about stimulation of human neural stem cells in vitro and in vivo.

The first suggestions that new neurons were born in the adult mammalian brain came from studies performed in the 1960s (Altman and Das 1965; Altman and Das 1967). It however took another three decades and refined technical procedures to overthrow the dogma that neurogenesis within the mammalian CNS is restricted to embryogenesis and the perinatal period (for review see (Momma, Johansson et al. 2000); (Kuhn and Svendsen 1999)). Treatment of neural disease and injury traditionally focuses on keeping existing neurons alive, but possibilities now arise for exploiting neurogenesis for therapeutic treatments of neurological disorders and diseases.

The source of new neurons is neural stem cells (NSC), located within the ependymal and/or subventricular zone (SVZ) lining the lateral ventricle (Doetsch, Caille et al. 1999; Johansson, Momma et al. 1999) and in the dentate gyrus of the hippocampus formation (Gage, Kempermann et al. 1998). Recent studies reveal the potential for several additional locations of NSC within the adult CNS (Palmer, Markakis et al. 1999). Asymmetric division of NSC maintain their number while generating a population of rapidly dividing precursor or progenitor cells (Johansson, Momma et al. 1999). The progenitors respond to a range of cues that dictate the extent of their proliferation and their fate, both in terms of the cell type that they differentiate into and the position that they ultimately take up in the brain.

The NSC of the ventricular system in the adult are likely counterparts of the embryonic ventricular zone stem cells lining the neural tube whose progeny migrate away to form the CNS as differentiated neurons and glia (Jacobson 1991). NSC persist in the adult lateral ventricle wall (LVW), generating neuronal progenitors which migrate down the rostral migratory stream to the olfactory bulb, where they differentiate into granule cells and periglomerular neurons (Lois and Alvarez-Buylla 1993). Substantial neuronal death occurs in the olfactory bulb generating a need for continuous replacement of lost neurons, a need satisfied by the migrating progenitors derived from the LVW (Biebl, Cooper et al. 2000). Further to this ongoing repopulation of olfactory bulb neurons, there are forceful indications that lost neurons from other brain regions can be replaced by progenitors from the LVW that differentiate into the lost neuron phenotype complete with appropriate neuronal projections and synapses with the correct target cell type (Snyder, Yoon et al. 1997; Magavi, Leavitt et al. 2000).

In vitro cultivation techniques have been established to identify the external signals involved in the regulation of NSC proliferation and differentiation (Johansson, Momma et al. 1999; Johansson, Svensson et al. 1999). The mitogens EGF and basic FGF allow neural progenitors, isolated from the ventricle wall and hippocampus, to be greatly expanded in culture (McKay 1997; Johansson, Svensson et al. 1999). The dividing progenitors remain in an undifferentiated state growing into large balls of cells known as neurospheres. Withdrawal of the mitogens combined with addition of serum induces differentiation of the progenitors into the three cell lineages of the brain: neurons, astrocytes and oligodendrocytes (Doetsch, Caille et al. 1999; Johansson, Momma et al. 1999). Application of specific growth factors can distort the proportions of each cell type in one way or another. For example, CNTF acts to direct the neural progenitors to an astrocytic fate (Johe, Hazel et al. 1996; Rajan and McKay 1998), while the thyroid hormone, triiodothyronine (T3) has been shown to promote oligodendrocyte differentiation (Johe, Hazel et al. 1996). Enhancement of neuronal differentiation of neural progenitors by PDGF has also been documented (Johe, Hazel et al. 1996; Williams, Park et al. 1997).

The ability to expand neural progenitor cells and then manipulate their cell fate has also had enormous implications in transplant therapies for neurological diseases in which specific cell types are lost. The most obvious example is Parkinson's Disease (PD) which is characterized by degeneration of dopaminergic neurons in the substantia nigra. Previous transplantation treatments for PD patients have used fetal tissue taken from the ventral midbrain at a time when substantia nigral dopaminergic neurons are undergoing terminal differentiation (Herman and Abrous 1994). The cells are grafted onto the striatum where they form synaptic contacts with host striatal neurons, their normal synaptic target, restoring dopamine turnover and release to normal levels with significant functional benefits to the patient (Herman and Abrous 1994) (for review see (Bjorklund and Lindvall 2000)). Grafting of fetal tissue is hindered by lack of donor tissue. In vitro expansion and manipulation of NSC, however, can potentially provide a range of well characterized cells for transplant-based strategies for neurodegenerative diseases, such as dopaminergic cells for PD. To this aim, the identification of factors and pathways that govern the proliferation and differentiation of neural cell types will prove fundamental.

Ultimately the identification of these proliferative and differentiating factors is likely to provide insights into the stimulation of endogenous neurogenesis for the treatment of neurological diseases and disorders. Intraventricular infusion of both EGF and basic FGF have been shown to proliferate the ventricle wall cell population, and in the case of EGF, extensive migration of progenitors into the neighboring striatal parenchyma (Craig, Tropepe et al. 1996; Kuhn, Winkler et al. 1997). The progenitors differentiated predominantly into a glial lineage while reducing the generation of neurons (Kuhn, Winkler et al. 1997). A recent study found that intraventricular infusion of BDNF in adult rats stimulates an increase in the number of newly generated neurons in the olfactory bulb and rostral migratory stream, and in parenchymal structures, including the striatum, septum, thalamus and hypothalamus (Pencea, Bingaman et al. 2001). These studies demonstrate that the proliferation of progenitors within the SVZ of the LVW can be stimulated and that their lineage can be manipulated to neuronal and glial fates. Currently the number of factors known to affect neurogenesis in vivo is small and their effects are either undesired or limited.

Therefore, there is a long felt need to identify other factors that can selectively stimulate neural stem cell activity through proliferation of neural progenitors and differentiation into the desired neuronal cell type. This activity would be beneficial for both stimulation of in vivo neurogenesis and culture of cells for transplantation therapy. The present invention demonstrates a role for PACAP, Maxadilan, VIP and their signaling pathways in the proliferation, differentiation, survival and migration of neural stem cells in vitro and in vivo.

### SUMMARY OF THE INVENTION

This invention relates generally to methods of influencing central nervous system cells to produce progeny that can replace damaged or missing neurons or other central nervous system cell types.

In one aspect, the invention includes a method of alleviating a symptom of a disorder of the nervous system comprising administering PACAP, Maxadilan, VIP or a combination thereof to modulate neural stem cell or neural progenitor cell activity in vivo to a patient suffering from the disease or disorder of the nervous system. For the purposes of this disclosure, disorder and disease shall have the same meaning.

In another aspect, the invention provides a method of modulating a PACAP receptor, a Maxadilan receptor, a VIP receptor or a combination thereof, on a neural stem cell or neural progenitor cell, the method comprising exposing the cell expressing the receptor to exogenous reagent, antibody, or affibody, wherein the exposure induces or inhibits the neural stem cell or neural progenitor cell to proliferate, differentiate or survive.

In a further aspect, the invention includes a method for reducing a symptom of a disease or disorder of the central nervous system in a mammal in need of such treatment comprising administering PACAP, Maxadilan, VIP, PACAP receptor agonist, Maxadilan receptor agonist or VIP receptor agonist to the mammal.

In another aspect, the invention provides a method for inducing the in situ proliferation, migration, differentiation or survival of a neural stem cell or neural progenitor cell located in the neural tissue of a mammal, the method comprising administering a therapeutically effective amount of PACAP, Maxadilan, VIP to the neural tissue to modulate the proliferation, migration differentiation or survival of the cell.

In another aspect, the invention includes a method for accelerating the growth of neural stem cells or neural progenitor cells in a desired target tissue in a subject, comprising administering to the subject an expression vector containing a PACAP, Maxadilan, or VIP gene in a therapeutically effective amount.

In another aspect, the invention includes a method of enhancing neurogenesis in a patient suffering from a disease or disorder of the central nervous system, by infusion of PACAP, Maxadilan, VIP, PACAP receptor agonist, Maxadilan receptor agonist, or VIP receptor agonist.

In a further aspect, the invention provides a method of alleviating a symptom in a patient suffering from a disease or disorder of the central nervous system by enhancing neurogenesis through infusion of PACAP, Maxadilan, VIP, PACAP receptor agonist, Maxadilan receptor agonist, or VIP receptor agonist.

In another aspect, the invention provides a method for producing a population of cells enriched for human neural stem cells or human neural progenitor cells, comprising: (a) contacting a population containing neural stem cells or neural progenitor cells with a reagent that recognizes a determinant on a PACAP receptor, Maxadilan receptor or VIP receptor; and (b) selecting for cells in which there is contact between the reagent and the determinant on the surface of the cells of step (a), to produce a population highly enriched for central nervous system stem cells. In one embodiment of the invention, the reagent is selected from the group consisting of a soluble receptor, a small molecule, a peptide, an antibody and an affibody. In another embodiment of the invention, the soluble receptor is a PACAP, Maxadilan, VIP receptor.

In a further aspect, the invention includes an in vitro cell culture comprising a cell population generated by the method previously described wherein the cell population is enriched in receptor expressing cells wherein the receptors are selected from the group consisting of PACAP receptor, Maxadilan receptor or VIP receptor.

In one aspect, the invention includes a method for alleviating a symptom of a disease or disorder of the central nervous system comprising administering the population of cells described above to a mammal in need thereof. In a further aspect, the invention includes a non-human mammal engrafted with the human neural stem cells or neural progenitor cells previously described. In a preferred embodiment of the invention, the non-human mammal is selected from the group including rat, mouse, rabbit, horse, sheep, pig and guinea pig.

In another aspect, the invention includes a method of reducing a symptom of a disease or disorder of the central nervous system in a subject comprising the steps of administering into the spinal cord of the subject a composition comprising a population of isolated neural stem cells or neural progenitor cells obtained from fetal or adult tissue; and PACAP, Maxadilan, VIP, PACAP receptor agonist, Maxadilan receptor agonist, or VIP receptor agonist or a combination thereof such that the symptom is reduced.

In another aspect, the invention includes a method of gene delivery and expression in a target cell of a mammal, comprising the step of introducing a viral vector into the target cell, wherein the viral vector has at least one insertion site containing a nucleic acid which encodes PACAP, Maxadilan or VIP, a PACAP receptor, a Maxadilan receptor or a VIP receptor, the nucleic acid gene operably linked to a promoter capable of expression in the host. In one embodiment of the invention, the viral vector is a non-lytic viral vector.

In another aspect, the invention includes a method of gene delivery and expression in a target cell of a mammal comprising the steps of: (a) providing an isolated nucleic acid fragment of a nucleic acid sequence which encodes for PACAP, Maxadilan or VIP, a PACAP receptor, a Maxadilan receptor or a VIP receptor; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the gene is expressed at detectable levels. In one embodiment of the invention, the virus is selected from the group consisting of retrovirus, adenovirus, pox virus, iridoviruses, coronaviruses, togaviruses, caliciviruses, lentiviruses, adeno-associated viruses and picomaviruses. In another embodiment of the invention, the pox virus is vaccinia. In another embodiment of the invention, the virus is a strain that has been genetically modified or selected to be non-virulent in a host.

In a further aspect, the invention includes a method for alleviating a symptom of a disease or disorder of the central nervous system in a patient comprising the steps of: (a) providing a population of neural stem cells or neural progenitor cells; (b) suspending the neural stem cells or neural progenitor cells in a solution comprising a mixture comprising PACAP, Maxadilan or VIP to generate a cell suspension; (c) delivering the cell suspension to an injection site in the central nervous system of the patient to alleviate the symptom. In one embodiment of the invention, the method described further comprises the step of injecting the injection site with a growth factor for a period of time before the step of delivering the cell suspension. In another embodiment of the invention, the method described further comprises the step of injecting the injection site with the growth factor after the delivering step.

In a further aspect, the invention includes a method for transplanting a population of cells enriched for human neural stem cells or human neural progenitor cells, comprising: (a) contacting a population containing neural stem cells or neural progenitor cells with a reagent that recognizes a determinant on a PACAP receptor, Maxadilan receptor or VIP receptor; (b) selecting for cells in which there is contacted between the reagent and the determinant on the surface of the cells of step (a), to produce a population highly enriched for central nervous system stem cells; and (c) implanting the selected cells of step (b) into a non-human mammal.

In a further aspect, the invention includes a method of modulating PACAP, Maxadilan or VIP receptor or a PACAP, Maxadilan or VIP ligand on the surface of a neural stem cell or neural progenitor cell comprising the step of exposing the cell expressing the receptor, or ligand to exogenous reagent, antibody, or affibody, wherein the exposure induces the neural stem cell or neural progenitor cell to proliferation, differentiation or survival. In one embodiment of the invention the neural stem cell or neural progenitor cell is derived from fetal brain, adult brain, neural cell culture or a neurosphere.

In a further aspect, the invention includes a method of determining an isolated candidate PACAP, Maxadilan or VIP receptor modulator compound for its ability to modulate neural stem cell or neural progenitor cell activity comprising the steps of: (a) administering the isolated candidate compound to a non-human mammal; and (b) determining if the candidate compound has an effect on modulating the neural stem cell or neural progenitor cell activity in the non-human mammal. In one embodiment of the invention, the determining step comprises comparing the neurological effects of the non-human mammal with a referenced non-human mammal not administered the candidate compound. In a further embodiment of the invention, the compound is selected from the group consisting of a peptide, a small molecule, and a receptor agonist. The neural stem cell or neural progenitor cell activity could be proliferation, differentiation, migration or survival.

### BRIEF DESCRIPTION OF THE DRAWINGS

FIG. 1 represents brightfield and darkfield micrographs of adcyaplrl mRNA positive cells in coronal and sagittal sections of adult mouse brain using a probe specific for all known mouse adcyaplrl isoforms.

FIG. 2 represents low magnification photomicrographs of adcyaplr mRNA expression using a probe specific for all known isoforms of the gene, and probes specific for the hop1/2 isoform and short isoform expression in coronal and sagittal sections of adult mouse brain.

FIG. 3 represents high magnification micrographs of adcyaplrl mRNA positive cells in the subventricular zone of human lateral ventricle wall and human hippocampal dentate gyrus.

FIG. 4 shows that adcyaplrl gene is expressed in cultured adult mouse neural stem cells.

FIG. 5 shows that the short isoform and hop1/2 isoforms of the adcyaplrl gene are expressed in cultured adult mouse neural stem cells.

FIG. 6 shows that the adcyaplrl gene is expressed in cultured adult human neural stem cells.

FIG. 7 shows that PACAP stimulates adult mouse NSC proliferation in non-adherent culture conditions.

FIG. 8 shows that Maxadilan stimulates adult mouse NSC proliferation in non-adherent culture conditions

FIG. 9 shows that PACAP and EGF synergistically proliferate adult mouse NSC in vitro.

FIG. 10 shows that PACAP and VEGF have an additive effect on adult mouse NSC number in vitro.

FIG. 11 shows that PACAP stimulation in adult mouse NSC proliferation is inhibited by PLC and PKC inhibitors but not PKA inhibition.

FIG. 12 shows that PACAP stimulates CREB phosphorylation in adult mouse and adult human NSC

FIG. 13 shows that PACAP stimulates AP-1 transcription through the MEK signaling pathway

FIG. 14 shows that VIP stimulates adult mouse NSC proliferation in vitro.

FIG. 15 shows that PACAP stimulates primary adult mouse NSC proliferation and neurosphere formation in vitro.

FIG. 16 shows that adult mouse NSC which proliferate after PACAP treatment retain multipotentiality, forming neurons (β-III Tubulin (A)), astrocytes (GFAP (B)) and oligodendrocytes (CNPase (C)).

FIG. 17 shows that PACAP promotes survival of cells derived from adult mouse NSC in vitro.

FIG. 18 shows that PACAP stimulates proliferation of adult mice subventricular zone NSC in vivo.

FIG. 19 shows that PACAP stimulates proliferation of adult mice hippocampal NSC/neural progenitors cells in vivo

FIG. 20 shows that PACAP stimulates adult mice hippocampal neurogenesis in vivo.

### DETAILED DESCRIPTION OF THE INVENTION

It has been discovered that certain reagents are capable of modulating the differentiation, migration, proliferation and survival of neural stem/progenitor cells both in vitro and in vivo. As used herein, the term “modulate” refers to having an affect in such a way as to alter the differentiation, migration, proliferation and survival of neural stem cell (NSC) or neural progenitor cell (NPC) activity. Since undifferentiated, pluripotent stem cells can proliferate in culture for a year or more, the invention described in this disclosure provides an almost limitless supply of neural precursors.

Throughout this disclosure, the term “neural stem cell” (NSC) includes “neural progenitor cell,” “neuronal progenitor cell,” “neural precurs cell,” and “neuronal precursor cell” (all referred to herein as NPC). NSC and NPC encompasses both a single cell or a plurality of cell (e.g., a cell population). These cells can be identified by their ability to undergo continuous cellular proliferation, to regenerate exact copies of themselves (self-renew), to generate a large number of regional cellular progeny, and to elaborate new cells in response to injury or disease. The term NPCs mean cells that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage. They also do not usually produce progeny of other embryonic germ layers when cultured by themselves in vitro unless dedifferentiated or reprogrammed in some fashion. As used herein, the term “neurosphere” refers to the ball of cells consisting of NSC.

As used herein, the term “reagent” refers to any substance that is chemically and biologically capable of activating a receptor, including peptides, small molecules, antibodies (or fragments thereof), affibodies and any molecule that dimerizes or multimerizes the receptors or replaces the need for activation of the extracellular domains. In one embodiment, the reagent is a small molecule.

As used herein, the term “antibody” or “immunoglobulin” as used in this disclosure refers to both polyclonal and monoclonal antibody and functional derivatives (i.e., engineered antibody) thereof. Antibodies can be whole inumunoglobulin of any class, e.g., IgG, IgM, IgA, IgD, IgE, or hybrid antibodies with dual or multiple antigen or epitope specificities, or fragments, e.g., F(ab′)2, F(ab)2, Fab′, Fab1 and the like, including hybrid fragments. Functional derivatives include engineered antibodies. The ambit of the term deliberately encompasses not only intact immunoglobulin molecules, but also such fragments and derivatives of immunoglobulin molecules (such as single chain Fv constructs, diabodies and fusion constructs) as may be prepared by techniques known in the art, and retaining a desired antibody binding specificity. The term “affibody” (U.S. Pat. No. 5,831,012) refers to highly specific affinity proteins that can be designed to bind to any desired target molecule. These antibody mimics can be manufactured to have the desired properties (specificity and affinity), while also being highly robust to withstand a broad range of analytical conditions, including pH and elevated temperature. The specific binding properties that can be engineered into each capture protein allow it to have very high specificity and the desired affinity for a corresponding target protein. A specific target protein will thus bind only to its corresponding capture protein. The small size (only 58 amino acids), high solubility, ease of further engineering into multifunctional constructs, excellent folding and absence of cysteines, as well as a stable scaffold that can be produced in large quantities using low cost bacterial expression systems, make affibodies superior capture molecules to antibodies or antibody fragments, such as Fab or single chain Fv (scFv) fragments, in a variety of Life Science applications. The term antibodies also encompasses engineered antibodies.

As used herein, the term “engineered antibody” encompasses all biochemically or recombinantly produced functional derivatives of antibodies. A protein is a functional derivative of an antibody if it has at least one antigen binding site (ABS) or a complementarity-determining region (CDR) that when combined with other CDR regions (on the same polypeptide chain or on a different polypeptide chain) can form an ABS. The definition of engineered antibody would include, at least, recombinant antibodies, tagged antibodies, labeled antibodies, Fv fragments, Fab fragments, recombinant (as opposed to natural) multimeric antibodies, single chain antibodies, diabodies, triabodies, tetravalent multimers (dimer of diabodies), pentavalent multimers (dimer of diabody and triabody), hexavalent multimers (dimer of triabodies) and other higher multimeric forms of antibodies.

The terms “recombinant nucleic acid” or “recombinantly produced nucleic acid” refer to nucleic acids such as DNA or RNA which has been isolated from its native or endogenous source and modified either chemically or enzymatically by adding, deleting or altering naturally-occurring flanking or internal nucleotides. Flanking nucleotides are those nucleotides which are either upstream or downstream from the described sequence or sub-sequence of nucleotides, while internal nucleotides are those nucleotides which occur within the described sequence or subsequence.

The term “recombinant means” refers to techniques where proteins are isolated, the cDNA sequence coding the protein identified and inserted into an expression vector. The vector is then introduced into a cell and the cell expresses the protein. Recombinant means also encompasses the ligation of coding or promoter DNA from different sources into one vector for expression of a PPC, constitutive expression of a protein, or inducible expression of a protein.

The term “promoter” refers to a DNA sequence which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent if the promoter is a constitutive promoter.

The term “enhancer” refers to a promoter element. An enhancer can increase the efficiency with which a particular gene is transcribed into mRNA irrespective of the distance or orientation of the enhancer relative to the start site of transcription.

“Complementary DNA (cDNA)” refers to a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complement.

“Expression” refers to the process by which a polypeptide is produced from a structural gene. The process involves transcription of the gene into mRNA and the translation of such mRNA into polypeptide(s).

“Cloning vector” refers to a DNA molecule, such as a plasmid, cosmid, phagemid, or bacteriophage, which has the capability of replicating autonomously in a host cell and which is used to transform cells for gene manipulation. Cloning vectors typically contain one or a small number of restriction endonuclease recognition sites at which foreign DNA sequences may be inserted in a determinable fashion without loss of an essential biological function of the vector, as well as a marker gene which is suitable for use in the identification and selection of cells transformed with the cloning vector. Marker genes typically include genes that provide tetracycline resistance or ampicillin resistance.

“Expression vector” refers to a DNA molecule comprising a cloned structural gene encoding a foreign protein which provides the expression of the foreign protein in a recombinant host. Typically, the expression of the cloned gene is placed under the control of (i.e., operably linked to) certain regulatory sequences such as promoter and enhancer sequences. Promoter sequences may be either constitutive or inducible.

“Recombinant Host” refers to a prokaryotic or eukaryotic cell which contains either a cloning vector or expression vector. This term is also meant to include those prokaryotic or eukaryotic cells that have been genetically engineered to contain the cloned gene(s) in the chromosome or genome of the host cell. The host cell is not limited to a unicellular organism. Multicellular organisms such as mammals, insects, and plants are also contemplated as host cells in the context of this invention. For examples of suitable hosts, see Sambrook et al., MOLECULAR CLONING: A LABORATORY MANUAL, Second Edition, Cold Spring Harbor Laboratory, Cold Spring Harbor, N.Y. (1989).

The term “treating” in its various grammatical forms in relation to the present invention refers to preventing, curing, reversing, attenuating, alleviating, minimizing, suppressing or halting the deleterious effects of a disease state, disease progression, disease causative agent (e.g., bacteria or viruses) or other abnormal condition.

The terms “recombinant protein,” “recombinantly produced protein” refer to a peptide or protein produced using non-native cells that do not have an endogenous copy of DNA able to express the protein. The cells produce the protein because they have been genetically altered by the introduction of the appropriate nucleic acid sequence. The recombinant protein will not be found in association with proteins and other subcellular components normally associated with the cells producing the protein.

According to the specific case, the “therapeutically effective amount” of an agent should be determined as being the amount sufficient to improve the symptoms of the patient in need of treatment or at least to partially arrest the disease and its complications. Amounts effective for such use will depend on the severity of the disease and the general state of the patient's health. Single or multiple administrations may be required depending on the dosage and frequency as required and tolerated by the patient.

The terms “binding specificity,” “specifically binds to” or “specifically immunoreactive with,” when referring to a protein, antibody, or antibody binding site (ABS) of the invention, refers to a binding reaction which is determinative of the presence of the protein or carbohydrate in the presence of a heterogeneous population of proteins and other biologics. A variety of immunoassay formats may be used to determine binding. For example, solid-phase ELISA immunoassays are routinely used to select antibodies specifically immunoreactive with a protein or carbohydrate. See Harlow & Lane, Antibodies, A Laboratory Manual, Cold Spring Harbor Publication, New York (1988) for a description of immunoassay formats and conditions that can be used to determine specific immunoreactivity.

The terms “nucleic acid encoding” or “nucleic acid sequence encoding” refer to a nucleic acid which directs the expression of a specific protein or peptide. The nucleic acid sequences include both the DNA strand sequence that is transcribed into RNA and the RNA sequence that is translated into protein. The nucleic acid sequences include both full length nucleic acid sequences as well as shorter sequences derived from the full length sequences. It is understood that a particular nucleic acid sequence includes the degenerate codons of the native sequence or sequences which may be introduced to provide codon preference in a specific host cell. The nucleic acid includes both the sense and antisense strands as either individual single strands or in the duplex form.

“Pharmaceutical composition” refers to formulations of various preparations. Parenteral formulations are known and are preferred for use in the invention. The formulations containing therapeutically effective amounts of the immunotoxins are either sterile liquid solutions, liquid suspensions or lyophilized versions and optionally contain stabilizers or excipients. Lyophilized compositions are reconstituted with suitable diluents, e.g., water for injection, saline, 0.3% glycine and the like, at a level of about from 0.01 mg/kg of host body weight to 10 mg/kg or more.

Preferred reagents of the invention include members of the VIP/secretin/glucagon family of peptides, such as the PACAP peptides, PACAP38 and PACAP27, that share an identical 27-aa N terminus and are alternatively processed from a 176-aa precursor called preproPACAP (Arimura 1998; Vaudry, Gonzalez et al. 2000). Vasoactive intestinal peptide (VIP) is a 28-amino acid peptide with a variety of actions in the central nervous system (CNS) and in several peripheral organs (Gressens 1999). VIP can bind specifically and with high affinity to VIPR1 and 2, but with 100-1000 lower affinity to ADCYAP1R1 (Jaworski and Proctor 2000).

The invention provides a method for in vivo modulation of PACAP activity, and for therapeutic administration of PACAP and Maxadilan peptides for drug screening. In one embodiment, the compounds above described are administered to neural tissue. In a preferred embodiment, the neural tissue is fetal or adult brain. In yet another embodiment, the population containing neural or neural-derived cells is obtained from a neural cell culture or neurosphere.

One receptor included in the present invention is ADCYAP1R1, a member of the VIP/secretin/glucagon receptor family, is included together with all isoforms (Jaworski and Proctor 2000)(see Example 7).

### PACAP Receptors and Their Ligands

The role of neuropeptides in neurogenesis and neuronal differentiation is emerging. In particular, although clear functional data are still missing, the pituitary adenylate cyclase-activating polypeptide (PACAP) and the vasoactive intestinal peptide (VIP) have been recently shown to have a potential role in these processes (Tyrrell and Landis 1994; Jaworski and Proctor 2000; Hansel, Eipper et al. 2001; Hansel, May et al. 2001). PACAP is a member of the VIP/secretin/glucagon family of peptides and exists in two amidated forms, PACAP38 and PACAP27, which share an identical 27-aa N terminus and are alternatively processed from a 176-aa precursor called preproPACAP (Arimura 1998; Vaudry, Gonzalez et al. 2000). The primary structure of PACAP38 has been conserved significantly during evolution from protochordates to mammals, suggesting that the peptide exerts important activities throughout the vertebrate phylum (Arimura 1998; Vaudry, Gonzalez et al. 2000). In Drosophila, recent molecular cloning and transgenic rescue experiments in the memory-mutant amnesiac, which has behavioral defects that include impaired olfaction-associated learning and changes in ethanol sensitivity, demonstrated that the amnesiac gene encodes a neuropeptide homologous to vertebrate PACAP (Feany and Quinn 1995; Moore, DeZazzo et al. 1998). In addition, mammalian PACAP activated both the cAMP and Ras/Raf signal-transduction pathways in Drosophila neurons, suggesting a neuromodulatory role of amnesiac (Drosophila PACAP) in specific neuronal populations (Zhong 1995). In mammals, PACAP occurs in neuronal elements, where it acts as a pleiotropic neuropeptide via three heptahelical G protein-linked receptors, one PACAP-specific (ADCYAP1R1) receptor and two receptors that it shares with VIP (VIPR1 and VIPR2). It is important to underline that VIP can also bind, although with lower affinity, the ADCYAP1R1 (Jaworski and Proctor 2000).

PACAP stimulates several different signaling cascades in neurons, leading to the activation of adenylate cyclase, phospholipase C, and mitogen-activated protein kinase and the mobilization of calcium (Hashimoto, Ishihara et al. 1993; Arimura 1998; Vaudry, Gonzalez et al. 2000). Histochemical studies have shown that PACAP immunoreactivity is observed in several regions of the central nervous system (CNS), including the dopamine (DA) and serotonin (5-HT) systems, with high concentrations found in the nucleus accumbens, hypothalamus, amygdala, substantia nigra, and dorsal raphe (Ghatei, Takahashi et al. 1993; Masuo, Suzuki et al. 1993; Piggins, Stamp et al. 1996). ADCYAP1R1 is also expressed throughout the target areas of both the mesocorticolimbic and nigrostriatal DA systems as well as 5-HT system (Hashimoto, Nogi et al. 1996). In addition, VIPR1 and VIPR2 also are expressed in these systems (Usdin, Bonner et al. 1994). These histochemical studies suggest a functional relationship between PACAP neurons and DA and 5-HT neurons. Pharmacological studies show that PACAP has neurotrophic and neuroprotective actions on mesencephalic DA neurons (Takei, Skoglosa et al. 1998), cortical neurons (Morio, Tatsuno et al. 1996), cerebellar granule cells (Villalba, Bockaert et al. 1997), and other neurons (Arimura 1998; Vaudry, Gonzalez et al. 2000). PACAP increases tyrosine hydroxylase activity in the nucleus accumbens (Moser, Scholz et al. 1999) and stimulates interleukin-6 production in astrocytes (Gottschall, Tatsuno et al. 1994). PACAP also is implicated in synaptic plasticity in the hippocampus (Gottschall, Tatsuno et al. 1994).

Of particular interest, ADCYAP1R1 is expressed during development at very high levels in ventricular zones through all the neuroaxis. In addition to the embryonic enrichment in proliferative zones, ADCYAP1R1 expression is maintained in areas of neurogenesis in the adult central nervous system, namely, the subventricular zone and in the hippocampal dentate gyrus (Jaworski and Proctor 2000) suggesting a pivotal role of PACAP in adult neurogenesis. Furthermore, it has been shown that VIP can stimulate proliferation during neurogenesis (Gressens, Paindaveine et al. 1997) suggesting that both neuropepetides, via their receptors, have an important role in modulating proliferation and differentiation at different stages of development of the CNS.

From a biochemical and molecular perspective, it is interesting to observe that PACAP and VIP pathways are principally mediated by elevated intracellular cAMP levels. This is in line with recent data that show that cAMP and cAMP response element-binding protein (CREB) play an important role in contributing to in vivo neurogenesis in the denate gyrus of the hippocampus (Nakagawa, Kim et al. 2002). However it has recently been shown that PKC can also activate CREB. Interestingly, PACAP can crosstalk to the PKC signaling pathway via a downstream effector of cAMP, such as RAP1, causing further activation of the CREB transcription factor (Vaudry, Stork et al. 2002).

### Maxadilan is a Specific ADCYAP1R1 Agonist

Maxadilan is a 63 amino acid peptide isolated from salivary gland extracts of the New World sand fly, Lutzomyia lougipalpis, with potent vasodilatory properties (Lemer, Ribeiro et al. 1991). Studies have demonstrated that unlike PACAP, which can bind both ADCYAP1R1 and VFPR1 & 2, Maxadilan binds specifically to only ADCYAP1R1 (Moro and Lerner 1997).

### Production of Reagents

Reagents for treatment of patients are recombinantly produced, purified and formulated according to well known methods.

Reagents of the invention and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The peptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of peptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, e.g., receptor binding, functional potency or duration of action. See, e.g., Doherty, et al., 1993. J. Med. Chem. 36: 2585-2594; Kirby, et al., 1993, J. Med. Chem. 36:3802-3808; Morita, et al., 1994, FEBS Lett. 353: 84-88; Wang, et al., 1993 Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the peptide backbone. This strategy can be used to develop peptide analogs of reagents with increased potency, selectivity and stability. A number of other methods have been used successfully to introduce conformational constraints into peptide sequences in order to improve their potency, receptor selectivity and biological half-life. These include the use of (i) C_{α}-methylamino acids (see, e.g., Rose, et al., Adv. Protein Chem. 37: 1-109 (1985); Prasad and Balaram, CRC Crit. Rev. Biochem., 16: 307-348 (1984)); (ii) N_{α}-methylamino acids (see, e.g., Aubry, et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh, et al., Biopolymers, 26: 819-829 (1987)). These and many other amino acid analogs are commercially available, or can be easily prepared. Additionally, replacement of the C-terminal acid with an amide can be used to enhance the solubility and clearance of a peptide.

Alternatively, a reagent may be obtained by methods well-known in the art for recombinant peptide expression and purification. A DNA molecule encoding the protein reagent can be generated. The DNA sequence is known or can be deduced from the protein sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequence, e.g., recognition sites for restriction enzymes which facilitate its cloning into a suitable cloning vector, such as a plasmid. Nucleic acids may be DNA, RNA, or a combination thereof. Nucleic acids encoding the reagent may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

Any of the methodologies known within the relevant art regarding the insertion of nucleic acid fragments into a vector may be used to construct expression vectors that contain a chimeric gene comprised of the appropriate transcriptional/translational control signals and reagent-coding sequences. Promoter/enhancer sequences within expression vectors may use plant, animal, insect, or fungus regulatory sequences, as provided in the invention.

A host cell can be any prokaryotic or eukaryotic cell. For example, the peptide can be expressed in bacterial cells such as E. coli, insect cells, fungi or mammalian cells (such as Chinese hamster ovary cells (CHO) or COS cells). Other suitable host cells are known to those skilled in the art. In one embodiment, a nucleic acid encoding a reagent is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195).

The host cells, can be used to produce (e.g., overexpress) peptide in culture. Accordingly, the invention further provides methods for producing the peptide using the host cells of the invention. In one embodiment, the method comprises culturing the host cell of invention (into which a recombinant expression vector encoding the peptide has been introduced) in a suitable medium such that peptide is produced. The method further involves isolating peptide from the medium or the host cell. Ausubel et al., (Eds). In: Current Protocols in Molecular Biology. J. Wiley and Sons, New York, N.Y. 1998.

An “isolated” or “purified” recombinant peptide or biologically active portion thereof is substantially free of cellular material or other contaminating proteins from the cell or tissue source from which the peptide of interest is derived. The language “substantially free of cellular material” includes preparations in which the peptide is separated from cellular components of the cells from which it is isolated or recombinantly produced. In one embodiment, the language “substantially free of cellular material” includes preparations of peptide having less than about 30% (by dry weight) of peptide other than the desired peptide (also referred to herein as a “contaminating protein”), more preferably less than about 20% of contaminating protein, still more preferably less than about 10% of contaminating protein, and most preferably less than about 5% contaminating protein. When the peptide or biologically active portion thereof is recombinantly produced, it is also preferably substantially free of culture medium, e.g., culture medium represents less than about 20%, more preferably less than about 10%, and most preferably less than about 5% of the volume of the peptide preparation.

The invention also pertains to variants of a reagent that function as either agonists (mimetics) or as antagonists. Variants of a reagent can be generated by mutagenesis, e.g., discrete point mutations. An agonist of a reagent can retain substantially the same, or a subset of, the biological activities of the naturally occurring form of the reagent. An antagonist of the reagent can inhibit one or more of the activities of the naturally occurring form of the reagent by, for example, competitively binding to the receptor. Thus, specific biological effects can be elicited by treatment with a variant with a limited function. In one embodiment, treatment of a subject with a variant having a subset of the biological activities of the naturally occurring form of the reagent has fewer side effects in a subject relative to treatment with the naturally occurring form of the reagent.

Preferably, the analog, variant, or derivative reagent is functionally active. As utilized herein, the term “functionally active” refers to species displaying one or more known functional attributes of a full-length reagent. “Variant” refers to a reagent differing from naturally occurring reagent, but retaining essential properties thereof. Generally, variants are overall closely similar, and in many regions, identical to the naturally occurring reagent.

Variants of the reagent that function as either agonists (mimetics) or as antagonists can be identified by screening combinatorial libraries of mutants of the reagent for peptide agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual peptides, or alternatively, as a set of larger fusion proteins (e.g., for phage display) containing the set of sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl. Acids Res. 11:477.

Derivatives and analogs of the reagent or individual moieties can be produced by various methods known within the art. For example, the polypeptide sequences may be modified by any number of methods known within the art. See e.g., Sambrook, et al., 1990. Molecular Cloning: A Laboratory Manual, 2nd ed., (Cold Spring Harbor Laboratory Press; Cold Spring Harbor, N.Y.). Modifications include: glycosylation, acetylation, phosphorylation, amidation, derivatization by known protecting/blocking groups, linkage to an antibody molecule or other cellular reagent, and the like. Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Derivatives and analogs may be full length or other than full length, if the derivative or analog contains a modified nucleic acid or amino acid, as described infra. Derivatives or analogs of the reagent include, but are not limited to, molecules comprising regions that are substantially homologous in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art (e.g., Wisconsin GCG software) or (iii) the encoding nucleic acid is capable of hybridizing to a sequence encoding the aforementioned peptides under stringent (preferred), moderately stringent, or non-stringent conditions. See, e.g., Ausubel, et al., Current Protocols in Molecular Biology, John Wiley and Sons, New York, N.Y., 1993.

Derivatives of the reagent may be produced by alteration of their sequences by substitutions, additions or deletions that result in functionally-equivalent molecules. One or more amino acid residues within the reagent may be substituted by another amino acid of a similar polarity and net charge, thus resulting in a silent alteration. Conservative substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

The reagent can be administered locally to any loci implicated in the CNS disorder pathology, e.g. any loci deficient in neural cells as a cause of the disease. For example, the reagent can be administered locally to the ventricle of the brain, substantia nigra, striatum, locus ceruleous, nucleus basalis of Meynert, pedunculopontine nucleus, cerebral cortex, spinal cord and retina.

Neural stem cells and their progeny can be induced to proliferate, differentiate, survive or migrate in vivo by administering to the host a reagent, alone or in combination with other agents, or by administering a pharmaceutical composition containing the reagent that will induce proliferation and differentiation of the cells. Pharmaceutical compositions include any substance that blocks the inhibitory influence and/or stimulates neural stem cells and stem cell progeny to proliferate, differentiate, migrate and/or survive. Such in vivo manipulation and modification of these cells allows cells lost, due to injury or disease, to be endogenously replaced, thus obviating the need for transplanting foreign cells into a patient.

### Antibodies

Included in the invention are antibodies to be used as reagents. The term “antibody” as used herein refers to immunoglobulin molecules and immunologically active portions of immunoglobulin (Ig) molecules, e.g., molecules that contain an antigen binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of PACAP, Maxadilan, VIP or a PACAP, Maxadilan, VIP receptor that is located on the surface of the protein, e.g., a hydrophilic region. A hydrophobicity analysis of the human those protein sequences will indicate which regions of the polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A PACAP, Maxadilan, VIP or ligand or receptor polypeptide or a fragment thereof comprises at least one antigenic epitope. An anti-PACAP, anti-Maxadilan, anti-VIP or anti-Protein A antibody of the present invention is said to specifically bind to the antigen when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

### Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the inmnunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

### Monoclonal Antibodies

The term “monoclonal antibody” (MAb) or “monoclonal antibody composition,” as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymnphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human mycloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subdloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the, coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

### Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intem. Rev. Inmunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ M as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymnology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA or TETA. Another bispecific antibody of interest binds the protein antigen described herein and further binds tissue factor (TF).

### Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

### Antibody Therapeutics

Antibodies of the invention, including polyclonal, monoclonal, humanized and fully human antibodies, may be used as therapeutic agents such as one of this invention. Such agents will generally be employed to treat or prevent a disease or pathology, specifically neurological disease, in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous PACAP, Maxadilan, VIP ligand to which it naturally binds. In this case, the antibody binds to the target and masks a binding site of the naturally occurring ligand, wherein the ligand serves as an effector molecule. Thus, the receptor mediates a signal transduction pathway for which ligand is responsible.

Alternatively, the effect may be one in which the antibody elicits a physiological result by virtue of binding to an effector binding site on the target molecule. In this case the target, a PACAP, Maxadilan, or VIP receptor having an endogenous ligand which needs to be modulated, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen and the rate at which an administered antibody is depleted from the free volume of the subject to which it is administered.

### Diseases and Disorders

Diseases and disorders that are characterized by altered (relative to a subject not suffering from the disease or disorder) levels or biological activity may be treated with therapeutics that antagonize (e.g., reduce or inhibit) or activate PACAP, Maxadilan or VIP activity. Therapeutics that antagonize activity may be administered in a therapeutic or prophylactic manner. Therapeutics that may be utilized include, but are not limited to: (i) an aforementioned peptide, analog, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (e.g., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (e.g., inhibitors, agonists and antagonists, including additional peptide mimetic of the invention or antibodies specific to a peptide of the invention) that alter the interaction between an aforementioned peptide and its binding partner.

Diseases and disorders that are characterized by altered (relative to a subject not suffering from the disease or disorder) levels or biological activity may be treated with therapeutics that increase (e.g., are agonists to) activity. In a preferred embodiment, the diseases to be treated include Alzheimer's disease, stroke, Parkinson's disease. Therapeutics that upregulate activity may be administered in a therapeutic or prophylactic manner. Therapeutics that may be utilized include, but are not limited to, an aforementioned peptide, analog, derivatives, fragments or homologs thereof, or an agonist that increases bioavailability.

Increased or decreased levels can be detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Therapeutic Methods

Another aspect of the invention pertains to methods of modulating PACAP, Maxadilan or VIP expression or activity for therapeutic purposes. In one embodiment, the modulatory method of the invention involves contacting a cell with an agent that modulates one or more of the activities of PACAP, Maxadilan, VIP. An agent that modulates this protein activity can be an agent as described herein, such as a nucleic acid or a protein, a naturally-occurring cognate ligand of a PACAP, Maxadilan or VIP receptor, a peptide, a PACAP, Maxadilan or VIP peptidomimetic, or other small molecule. In one embodiment, the agent stimulates the activity of the PACAP, Maxadilan or VIP signaling pathway. Examples of such stimulatory agents include active PACAP, Maxadilan or VIP protein and a nucleic acid molecule encoding PACAP, Maxadilan or VIP that has been introduced into the cell. These modulatory methods can be performed in vitro (e.g., by culturing the cell with the agent) or, alternatively, in vivo (e.g., by administering the agent to a subject). As such, the invention provides methods of treating an individual afflicted with a disease or disorder, specifically a neurological disorder. In one embodiment, the method involves administering a reagent (e.g., an reagent identified by a screening assay described herein), or combination of reagents that modulate (e.g., up-regulates or down-regulates) PACAP, Maxadilan or VIP expression or activity. In another embodiment, the method involves administering a PACAP, Maxadilan, VIP or nucleic acid molecule encoding said proteins as therapy to modulate proliferation, differentiation, migration and/or survival of NSC.

Stimulation of PACAP, Maxadilan or VIP activity is desirable in situations in which PACAP, Maxadilan or VIP are abnormally downregulated and/or in which increased PACAP, Maxadilan or VIP activity is likely to have a beneficial effect. One example of such a situation is where a subject has a disorder characterized by aberrant cell proliferation and/or differentiation (e.g., Parkinson's disease and Alzheimer's disease).

### Determination of the Biological Effect of the Therapeutic

In various embodiments of the invention, suitable in vitro or in vivo assays are performed to determine the effect of a specific therapeutic and whether its administration is indicated for treatment of the affected tissue.

In various specific embodiments, in vitro assays may be performed with representative stem cells or newly differentiated cells involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Pharmaceutical Compositions

The invention provides methods of influencing central nervous system cells to produce progeny that can replace damaged or missing neurons in the central nervous system or other central nervous system cell types by exposing a patient, suffering from a neurological disease or disorder, to a reagent (e.g. PACAP, Maxadilan, VIP) in a suitable formulation through a suitable route of administration, that modulates NSC or NPC activity in vivo. In all embodiment of the inventions, the reference to disease or disorder of the nervous system may include any disorder and, for example, at least the following disorders: neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders. In one embodiment of the invention, the disease or disorder of the nervous system is selected from the group consisting of Parkinson's disease and Parkinsonian disorders, Huntington's disease, Alzheimer's disease, Amyotrophic Lateral Sclerosis, spinal ischemia, ischemic stroke, spinal cord injury and cancer-related brain/spinal cord injury. In a further embodiment of the invention, the disease or disorder of the nervous system is selected from the group consisting of schizophrenia and other psychoses, lissencephaly syndrome, depression, bipolar depression/disorder, anxiety syndromes/disorders, phobias, stress and related syndromes, cognitive function disorders, aggression, drug and alcohol abuse, obsessive compulsive behaviour syndromes, seasonal mood disorder, borderline personality disorder, cerebral palsy, life style drug, multi-infarct dementia, Lewy body dementia, age related/geriatric dementia, epilepsy and injury related to epilepsy, temporal lobe epilepsy, spinal cord injury, brain injury, brain surgery, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy, sleep disorders, and disorders of cognitive performance or memory.

This invention provides a method of treating a neurological disease or disorder comprising administering a reagent that modulates neural stem cell or neural progenitor cell activity in vivo to a mammal. The term “mammal” refers to any mammal classified as a mammal, including humans, cows, horses, dogs, sheep and cats. In one embodiment, the mammal is a human.

The invention provides a regenerative cure for neurodegenerative diseases by stimulating ependymal cells and subventricular zone cells to proliferate, migrate, differentiate and survive into the desired neural phenotype targeting loci where cells are damaged or missing. In vivo stimulation of ependymal stem cells is accomplished by locally administering a reagent to the cells in an appropriate formulation. By increasing neurogenesis, damaged or missing neurons can be replaced in order to enhance brain function in diseased states.

A pharmaceutical composition useful as a therapeutic agent for the treatment of central nervous system disorders is provided. For example, the composition includes a reagent of the invention, which can be administered alone or in combination with the systemic or local co-administration of one or more additional agents. Such agents include preservatives, ventricle wall permeability increasing factors, stem cell mitogens, survival factors, glial lineage preventing agents, anti-apoptotic agents, anti-stress medications, neuroprotectants, and anti-pyrogenics. The pharmaceutical composition preferentially treats CNS diseases by stimulating cells (e.g., ependymal cells and subventricular zone cells) to proliferate, migrate and differentiate into the desired neural phenotype, targeting loci where cells are damaged or missing.

A method for treating a subject suffering from a CNS disease or disorder is also provided. This method comprises administering to the subject an effective amount of a pharmaceutical composition containing a reagent (1) alone in a dosage range of 0.001 ng/kg/day to 10 mg/kg/day, preferably in a dosage range of 0.01 ng/kg/day to 5 mg/kg/day, more preferably in a dosage range of 0.1 ng/kg/day to 1 mg/kg/day, most preferably in a dosage range of 0.1 ng/kg/day to 10 μg/kg/day, (2) in a combination with a ventricle wall permeability increasing factor, or (3) in combination with a locally or systemically co-administered agent.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that it can be handled with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., chimeric peptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Nucleic acid molecules encoding a proteinaceous agent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

In another embodiments, the reagent is administered in a composition comprising at least 90% pure reagent. The reagent can be, for example, PACAP, Maxadilan or VIP.

Preferably the reagent is formulated in a medium providing maximum stability and the least formulation-related side-effects. In addition to the reagent, the composition of the invention will typically include one or more protein carrier, buffer, isotonic salt and stabilizer.

In some instances, the reagent can be administered by a surgical procedure implanting a catheter coupled to a pump device. The pump device can also be implanted or be extracorporally positioned. Administration of the reagent can be in intermittent pulses or as a continuous infusion. Devices for injection to discrete areas of the brain are known in the art (see, e.g., U.S. Pat. Nos. 6,042,579; 5,832,932; and 4,692,147).

Reagents containing compositions can be administered in any conventional form for administration of a protein. A reagent can be administered in any manner known in the art in which it may either pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the agents to affect solubility or clearance of the peptide. Peptidic molecules may also be synthesized with D-amino acids to increase resistance to enzymatic degradation. In some cases, the composition can be co-administered with one or more solubilizing agents, preservatives, and permeation enhancing agents. Examples of pharmaceutically acceptable carriers include lactose, glucose, sucrose, sorbitol, mannitol, corn starch, crystalline cellulose, gum arabic, calcium phosphate, alginates, calcium silicate, microcrystalline cellulose, polyvinyl pyrrolidone, tragacanth gum, gelatin, syrup, methyl cellulose, carboxymethyl cellulose, methylhydroxybenzoic acid esters, propylhydroxybenzoic acid esters, talc, magnesium stearates, inert polymers, water and mineral oils.

For example, the composition can include a preservative or a carrier such as proteins, carbohydrates, and compounds to increase the density of the pharmaceutical composition. The composition can also include isotonic salts and redox-control agents.

In some embodiments, the composition administered includes the reagent and one or more agents that increase the permeability of the ventricle wall, e.g. “ventricle wall permeability enhancers.” Such a composition can help an injected composition penetrate deeper than the ventricle wall. Examples of suitable ventricle wall permeability enhancers include, for example, liposomes, VEGF (vascular endothelial growth factor), IL-s, TNFα, polyoxyethylene, polyoxyethylene ethers of fatty acids, sorbitan monooleate, sorbitan monolaurate, polyoxyethylene monolaurate, polyoxyethylene sorbitan monolaurate, fusidic acid and derivatives thereof, EDTA, disodium EDTA, cholic acid and derivatives, deoxycholic acid, glycocholic acid, glycodeoxycholic acid, taurocholic acid, taurodeoxycholic acid, sodium cholate, sodium glycocholate, glycocholate, sodium deoxycholate, sodium taurocholate, sodium glycodeoxycholate, sodium taurodeoxycholate, chenodeoxycholic acid, urosdeoxycholic acid, saponins, glycyrrhizic acid, ammonium glycyrrhizide, decamethonium, decamethonium bromide, dodecyltrimethylammonium bromide, and dimethyl-β-cyclodextrin or other cyclodextrins.

### Drug Screening

The invention also provides a method of using the receptors or receptor/reagent complexes for analyzing or purifying certain stem or progenitor cell populations, using e.g. antibodies, against the receptors or receptor/reagent complexes.

In another aspect, the invention provides a method for screening for reagents that influence stem and progenitor cells. In some applications, neural cells (undifferentiated or differentiated) are used to screen factors that promote maturation into neural cells, or promote proliferation and maintenance of such cells in long-term culture. For example, candidate reagents are tested by adding them to cells in culture at varying dosages, and then determining any changes that result, according to desirable criteria for further culture and use of the cells. Physical characteristics of the cells can be analyzed by observing cell and neurite growth with microscopy. The induction of expression of increased levels of proliferation, differentiation and migration can be analyzed with any technique known in the art which can identify proliferation and differentiation. Such techniques include RT-PCR, in situ hybridization, and ELISA.

In one aspect, novel receptor/reagents in undifferentiated neurospheres was examined using RT-PCR techniques. In particular, genes that are up-regulated in these undifferentiated neurospheres were identified. As used herein, the term “up-regulation” refers to a process that increases reagent/receptor interactions due to an increase in the number of available receptors. The presence of these genes suggests a potential role in the mediation of signal transduction pathways in the regulation of NSC function. Furthermore, by knowing the levels of expression of the receptors or their various reagents, it is possible to diagnose disease or determine the role of stem and progenitor cells in the disease. By analyzing the genetic or amino-acid sequence variations in these genes or gene products, it is possible to diagnose or predict the development of certain diseases. Such analysis will provide the necessary information to determine the usefulness of using stem or progenitor cell based treatments for disease.

In another aspect, in situ hybridization is performed on adult mouse brain sections to determine which cells in the adult brain express these signaling pathways. This data is helpful in determining treatment options for various neurological diseases.

To determine the effect of a potential reagent on neural cells, a culture of NSC derived from multipotent stem cells can be obtained from normal neural tissue or, alternatively, from a host afflicted with a CNS disease or disorder. The choice of culture will depend upon the particular agent being tested and the effects one wishes to achieve. Once the cells are obtained from the desired donor tissue, they are proliferated in vitro in the presence of a proliferation-inducing reagent.

The ability of various biological agents to increase, decrease or modify in some other way the number and nature of the stem cell progeny proliferated in the presence of the proliferative factor can be screened on cells proliferated by the methods previously discussed. For example, it is possible to screen for reagents that increase or decrease the proliferative ability of NSC which would be useful for generating large numbers of cells for transplantable purposes. In these studies precursor cells are plated in the presence of the reagent in question and assayed for the degree of proliferation and survival or progenitor cells and their progeny can be determined. It is possible to screen neural cells which have already been induced to differentiate prior to the screening. It is also possible to determine the effects of the reagent on the differentiation process by applying them to precursors cells prior to differentiation. Generally, the reagent will be solubilized and added to the culture medium at varying concentrations to determine the effect of the agent at each dose. The culture medium may be replenished with the reagent every couple of days in amounts so as to keep the concentration of the reagent somewhat constant.

Changes in proliferation are observed by an increase or decrease in the number of neurospheres that form and/or an increase or decrease in the size of the neurospheres, which is a reflection of the rate of proliferation and is determined by the numbers of precursor cells per neurosphere.

Using these screening methods, it is possible to screen for potential drug side-effects on prenatal and postnatal CNS cells by testing for the effects of the biological agents on stem cell and progenitor cell proliferation and on progenitor cell differentiation or the survival and function of differentiated CNS cells.

Other screening applications of this invention relate to the testing of pharmaceutical compounds for their effect on neural tissue. Screening may be done either because the compound is designed to have a pharmacological effect on neural cells, or because a compound designed to have effects elsewhere may have unintended side effects on the nervous system. The screening can be conducted using any of the neural precursor cells or terminally differentiated cells of the invention.

Effect of cell function can be assessed using any standard assay to observe phenotype or activity of neural cells, such as receptor binding, proliferation, differentiation, survival—either in cell culture or in an appropriate model.

### Therapeutic Uses

The fact that neural stem cells are located in the tissues lining ventricles of mature brains offers several advantages for the modification and manipulation of these cells in vivo and the ultimate treatment of various neurological diseases, disorders, and injury that affect different regions of the CNS. Therapy for these diseases can be tailored accordingly so that stem cells surrounding ventricles near the affected region would be manipulated or modified in vivo using the methods described herein. The ventricular system is found in nearly all brain regions and thus allows easier access to the affected areas. In order to modify the stem cells in vivo by exposing them to a composition comprising a reagent, it is relatively easy to implant a device that administers the composition to the ventricle and thus, to the neural stem cells. For example, a cannula attached to an osmotic pump may be used to deliver the composition. Alternatively, the composition may be injected directly into the ventricles. The neural stem cell progeny can migrate into regions that have been damaged as a result of injury or disease. Furthermore, the close proximity of the ventricles to many brain regions would allow for the diffusion of a secreted neurological agent by the stem cells or their progeny.

In an additional embodiment, a reagent of the invention is administered locally, as described above, in combination with an agent administered locally or systemically. Such agents include, for example, one or more stem cell mitogens, survival factors, glial-lineage preventing agents, anti-apoptotic agents, anti-stress medications, neuroprotectants, and anti-pyrogenics, or any combination thereof.

The agent is administered systemically before, during, or after administration of the reagent of the invention. The locally administered agent can be administered before, during, or after the reagent administration.

For treatment of Huntington's Disease, Alzheimer's Disease, Parkinson's Disease, and other neurological disorders affecting primarily the forebrain, a reagent alone or with an additional agent or agents is delivered to the ventricles of the forebrain to affect in vivo modification or manipulation of the stem cells. For example, Parkinson's Disease is the result of low levels of dopamine in the brain, particularly the striatum. It is therefore advantageous to induce a patient's own quiescent stem cells to begin to divide in vivo and to induce the progeny of these cells to differentiate into dopaminergic cells in the affected region of the striatum, thus locally raising the levels of dopamine.

Normally the cell bodies of dopaminergic neurons are located in the substantia nigra and adjacent regions of the mesencephalon, with the axons projecting to the striatum. The methods and compositions of the invention provide an alternative to the use of drugs and the controversial use of large quantities of embryonic tissue for treatment of Parkinson's disease. Dopamine cells can be generated in the striatum by the administration of a composition comprising a reagent of the invention to the lateral ventricle.

For the treatment of MS and other demyelinating or hypomyelinating disorders, and for the treatment of Amyotrophic Lateral Sclerosis or other motor neuron diseases, a reagent of the invention, alone or with an additional agent or agents is delivered to the central canal.

In addition to treating CNS tissue immediately surrounding a ventricle, a reagent of the invention, alone or with an additional agent or agents can be administered to the lumbar cistern for circulation throughout the CNS.

In other aspects, neuroprotectants can also be co-administered systemically or locally before, during and/or after infusion of a regent of the invention. Neuroprotectants include antioxidants (agents with reducing activity, e.g., selenium, vitamin E, vitamin C, glutathione, cysteine, flavinoids, quinolines, enzymes with reducing activity, etc), Ca-channel modulators, Na-channel modulators, glutamate receptor modulators, serotonin receptor agonists, phospholipids, unsaturated- and polyunsaturated fatty acids, estrogens and selective estrogen receptor modulators (SERMS), progestins, thyroid hormone and thyroid hormone-mimicking compounds, cyclosporin A and derivatives, thalidomide and derivatives, methylxanthines, MAO inhibitors; serotonin-, noradrenaline and dopamine uptake blockers; dopamine agonists, L-DOPA, nicotine and derivatives, and NO synthase modulators.

Certain reagents of the invention may be pyrogenic following IV injection (in rats; Am. J. Physiol. Regul. Integr. Comp. Physiol. 2000 278:R1275-81). Thus, in some aspects of the invention, antipyrogenic agents like cox2 inhibitors, indomethacin, salisylic acid derivatives and other general anti-inflammatory/anti-pyrogenic compounds can be systemically or locally administered before, during and/or after administration of the reagent of the invention.

In another aspect of the invention, anti-apoptotic agents including caspase inhibitors and agents useful for antisense-modulation of apoptotic enzymes and factors can be administered before, during, or after administration of the reagent of the invention.

Stress syndromes lower neurogenesis, therefore in some aspects, it may be desirable to treat a subject with anti-stress medications such as, e.g., anti-glucocorticoids (e.g., RU486) and beta-blockers, administered systemically or locally before, during and/or after infusion of the reagent of the invention.

Methods for preparing the reagent dosage forms are known, or will be apparent, to those skilled in this art.

The amount of reagent to be administered will depend upon the exact size and condition of the patient, but will be from 0.5 ng/kg/day to 500 ng/kg/day in a volume of 0.001 to 10 ml.

The duration of treatment and time period of administration of reagent will also vary according to the size and condition of the patient, the severity of the illness and the specific composition and method being used.

The effectiveness of each of the foregoing methods for treating a patient with a CNS disease or disorder is assessed by any known standardized test for evaluating the disease.

Specific Embodiments

One embodiment of the invention is directed to a method of alleviating a symptom of a disorder of the nervous system in a patient by administering a “NSC therapeutic agent” to the patient. The NSC therapeutic agent is may be PACAP, Maxadilan, PACAP receptor agonist, ADCYAP1R1 agonist or a combination of these agents. Administration of the NSC therapeutic agent modulates a NSC activity (proliferation, differentiation, migration, or survival) in vivo to alleviate the symptom.

The NSC therapeutic agent may be administered in a dose between 0.001 ng/kg/day to 10 mg/kg/day. Other suitable dosage ranges are: between 0.01 ng/kg/day to 5 mg/kg/day, between 0.1 ng/kg/day to 1 mg/kg/day, or between 0.1 ng/kg/day to 10 μg/kg/day.

Another method for determining proper dosage is to administering sufficient NSC therapeutic agents to achieve a target tissue concentration of 0.01 nM to 1 μM. The target tissue to be monitor could be any neural or CNS tissue, including, at least, the ventricular wall, the volume adjacent to the wall of the ventricular system, hippocampus, alveus, striatum, substantia nigra, retina, nucleus basalis of Meynert, spinal cord, thalamus, hypothalamus and cortex. Other suitable target tissue includes a region of tissue that is impaired by stroke injury or ischemic injury.

Administration of any of the NSC therapeutic agents, may be performed by injection. The method of injection include subcutaneous, intraperitoneal, intramusclular, intracerebroventricular, intraparenchymal, intrathecal or intracranial injection. In another embodiment, the NSC therapeutic agents may be administered orally. Other suitable administrations means include administeration to the buccal, nasal or rectal mucosa. In addition, NSC therapeutic agents may be administered by via peptide fusion or micelle delivery.

The disorders that can be treated by the methods of the invention includes any disorder listed in this disclosure. These disorders may be classified to include, at least, neurodegenerative disorders, NSC disorders, neural progenitor disorders, ischemic disorders, neurological traumas, affective disorders, neuropsychiatric disorders, degenerative diseases of the retina, retinal injury/trauma, cognitive performance and learning and memory disorders.

Another embodiment of the invention is directed to a method of modulating the activity of a receptor for PACAP, or Maxadilan on a NSC. The method involves exposing the cell expressing the receptor to a modulator agent so that the exposure induces NSC to proliferate, differentiate, migrate or survive. In this embodiment, the modulator agent may be an exogenous reagent, an antibody (monoclonal, polyclonal, or an engineered antibody), an affibody or a combination of these agents. The PACAP receptor, which is targeted for contact by the modulator agent may be ADCYAP1R1, VIPR1 or VIPR2. Similarly, the Maxadilan receptor may be ADCYAP1R1.

The modulator agent may be PACAP, Maxadilan, PACAP receptor agonist, or a ADCYAP1R1 agonist. Furthermore, the modulator agent may be pegylated to enhance its stability and efficacy in patients. Methods of pegylating proteins and reagents are well known to those of skill in the art and are described, for example, in U.S. Pat. Nos. 5,166,322, 5,766,897, 6,420,339 and 6,552,170.

In a preferred embodiment, the NSC is derived from fetal brain, adult brain, neural cell culture or a neurosphere. In another preferred embodiment, the NSC is derived from tissue enclosed by dura mater, peripheral nerves or ganglia. Other examples of suitable NSC include NSC derived from stem cells originating from pancreas, skin, muscle, adult bone marrow, liver, umbilical cord tissue or umbilical cord blood.

Another embodiment of the invention is directed to a method of stimulating mammalian adult NSC to proliferate, to undergo neurogenesis, to differentiate or to migrate. In the method, the adult NSC cells are contacted to PACAP, Maxadilan, PACAP receptor agonist, and ADCYAP1R1 agonist to form a treated NSC cell that has improved proliferation, neurogenesis, migration, or differentiation properties compared to untreated cells. The NSC cells may be derived from lateral ventricle wall of a mammalian brain. In a preferred embodiment, the NSC is derived from stem cells from pancreas, skin, muscle, adult bone marrow, liver, umbilical cord tissue or umbilical cord blood.

Another embodiment of the invention is directed to a method for synergistically stimulation of mammalian adult NSC proliferation and neurogenesis. In the method, a mammalian adult NSC is contacted to a growth factor and a NSC therapeutic agent. The two reagents induces the mammalian induce the NSC cell to proliferate at a rate greater than either the growth factor or NSC therapeutic agent alone. In a surprising discovery, the combination of growth factor and NSC therapeutic agent has a synergistic effect that is greater that the sum of growth factor effect and NSC therapeutic agent effect. In a preferred embodiment, the growth factor for use in this method is EGF.

Another embodiment of the invention is directed to a method for cooperative stimulation of mammalian adult NSC proliferation and neurogenesis. In the method, a mammalian adult NSC is contacted to a growth factor and a NSC therapeutic agent. The two reagents induces the mammalian induce the NSC cell to proliferate at a rate greater than either the growth factor or NSC therapeutic agent alone. In a preferred embodiment, the growth factor for use in this method is VEGF.

Another embodiment of the invention is directed to a method of stimulating mammalian adult NSC proliferation. The method involves contacting a NSC therapeutic agent to a mammalian adult NSC. The NSC therapeutic agent induces an increase in intracellular CREB phosphorylation. Mammalian adult NSC proliferation is, in turn, induced by the intracellular CREB phosphorylation.

Another embodiment of the invention is directed to a method of stimulating mammalian adult NSC proliferation. The method involves contacting a NSC therapeutic agent to a mammalian adult NSC. The NSC therapeutic agent induces an increase in intracellular AP-1 transcription. Mammalian adult NSC proliferation is, in turn, induced by the intracellular AP-1 transcription.

Another embodiment of the invention is directed to a method of stimulating mammalian adult NSC proliferation. The method involves contacting a NSC therapeutic agent to a mammalian adult NSC. The NSC therapeutic agent induces an increase in intracellular protein kinase C activity. Mammalian adult NSC proliferation is, in turn, induced by the intracellular protein kinase C activity.

Another embodiment of the invention is directed to a method for stimulating survival of mammalian adult NSC progeny by contacting the NSC cell with a NSC therapeutic agent. The increase in survival of NSC progeny may be characterized and predicted by (1) increased intracellular CREB phosphorylation, (2) increased intracellular AP-1 transcription, (3) increased intracellular protein kinase C activity and (4) increased intracellular protein kinase A activity. Mammalian adult NSC progeny survival can be mediated by stimulating any of the four characteristics listed above by the use of a NSC therapeutic agent.

Another embodiment of the invention is directed to a method of stimulating primary adult mammalian NSC to proliferate to form neurospheres. In the method, adult mammalian NSC is contacted with a NSC therapeutic agent to cause the cells to proliferate and form neurosphere.

Another embodiment of the invention is directed to a method for reducing a symptom of a central nervous system disorder in a mammal by administering a NSC therapeutic agent to the mammal. The agonist may be, for example, an antibody, an affibody, a small molecule, peptide and a receptor. The receptor may be a receptor for PACAP, Maxadilan or a ADCYAP1R1 receptor. In a preferred embodiment, the administration may be local or systemic. Further, the administration may include a ventricle wall permeability enhancer. The ventrical wall permeability enhancer may be administered before or after the NSC therapeutic agent. In a preferred embodiment, the NSC therapeutic agent is mixed with the permeability enhancer and a pharmaceutically acceptable carrier and administered. In a preferred embodiment, the method is enhanced by a further administration of stem cell mitogens, survival factors, glial-lineage preventing agents, anti-apoptotic agents, anti-stress medications, neuroprotectants, anti-pyrogenics, differentiation factors and a combination thereof.

Another embodiment of the invention is directed to a method for inducing the in situ proliferation, differentiation, migration or survival of a NSC located in the neural tissue of a mammal. The method involves administering a therapeutically effective amount of a NSC therapeutic agent to the neural tissue to induce the proliferation, differentiation, migration or survival of the NSC.

Another embodiment of the invention is directed to a method for accelerating the growth of NSC in a desired target tissue in a patient. In the method, a target tissue is transfected with an expression vector containing an open reading frame encoding PACAP, Maxadilan, VIPR1, VIPR2 or ADCYCAP1R1 gene in a therapeutically effective amount. The expression vector directs the expression of the open reading frame and the expressed protein accelerate the growth of the NSC in the target tissue. One advantage of this method is that while all, or most, of the cells in the targeted tissue is transfected, only the NSC cells are induced to accelerate the growth.

The transfection step may be performed by administration of the expression vector by injection. Any of the injection methods described in this disclosure may be used. These method includes, at least, subcutaneous, intraperitoneal, intramuscluar, intracerebroventricular, intraparenchymal, intrathecal or intracranial injection. The expression vector may be, for example, a non-viral expression vector encapsulated in a liposome.

Another embodiment of the invention is directed to a method of enhancing neurogenesis in a patient suffering from a central nervous system disorder by infusing a NSC therapeutic agent into the patient.

Another embodiment of the invention is directed to a method of alleviating a symptom of a central nervous system disorder in a patient by infusing PACAP, Maxadilan, PACAP receptor agonist, and ADCYAP1R1 agonist into the patient.

Another embodiment of the invention is directed to a method for producing a cell population enriched for human NSC. The method involves contacting a cell population with NSC with a reagent that specifically bind a determinant on a receptor for PACAP or Maxadilan. Then cells in which there is contact between the reagent and the determinant on the surface of the cells of the previous step is selected to produce a population highly enriched for central nervous system stem cells. The reagent may be a small molecule, a peptide, an antibody and an affibody. In one embodiment the population containing NSC are obtained from a neural tissue progenitor cell. A neural tissue progenitor cell is any population of cells which gives rise to neural tissue. For example, the cell population may be a cell population derived from whole mammalian fetal brain or whole mammalian adult brain. Further, the human NSC may be derived from stem cells originating from a tissue such as pancreas, skin, muscle, adult bone marrow, liver, umbilical cord tissue and umbilical cord blood. The described method is useful for enriching for cells expressing receptors such as ADCYAP1R1, VIPR1 or VIPR2.

Another embodiment of the invention is directed to an in vitro cell culture comprising a cell population generated by the method of the previous paragraph. Another embodiment of the invention is directed to a method for alleviating a symptom of a central nervous system disorder comprising administering the cells to a mammal exhibiting the symptom. A non-human mammal engrafted with the human NSC made by the method is also envisioned. The non-human mammal may be, for example, a rat, mouse, rabbit, horse, sheep, pig or guinea pig.

Another embodiment of the invention is directed to a method for reducing a symptom of a CNS disorder in a patient with the step of administering into the spinal cord of the patient a composition with a population of isolated NSC obtained from fetal or adult tissue; and a NSC therapeutic agent.

Another embodiment of the invention is directed to a method of reducing a symptom of a central nervous disorder in a patient. In the method, a viral vector for expressing a NSC therapeutic agent is introduced into a target cell. For expression, the viral vector may have at least one insertion site containing a nucleic acid which encoded a NSC therapeutic agent linked to a promoter capable of expression in the host cell (i.e., target cell). The NSC therapeutic agent is expressed to produce a protein in a target cell to reduce said symptom. In a preferred embodiment, the viral vector is a non-lytic viral vector.

Another embodiment of the invention is directed to a method of gene delivery and expression in a target cell of a mammal. The method comprise providing a nucleic acid molecule encoding a NSC therapeutic agent, selecting a viral vector with for insertion of the isolated nucleic acid molecule so that the molecule can be operably linked to a promoter capable of expression in the target cells, inserting the isolated nucleic acid fragment into the insertion site, and introducing the vector into the target cell wherein the gene is expressed at detectable levels.

The virus may be, for example, a retrovirus, adenovirus, pox virus (vaccinia), iridoviruses, coronaviruses, togaviruses, caliciviruses, lentiviruses, adeno-associated viruses or picornaviruses. In a preferred embodiment, the virus strain is genetically modified to be non-virulent in a host.

Another embodiment of the invention is directed to a method for alleviating a symptom of a disorder of the nervous system with the steps of providing a population of NSC, suspending the NSC in a solution comprising PACAP or Maxadilan or a combination thereof to create a cell suspension, and delivering the cell suspension to an injection site in the nervous system of the patient to alleviate the symptom. In addition, a further step of administering to the injection site a growth factor for a period of time before or after the step of delivering the cell suspension may be added.

Another embodiment of the invention is directed to a method for transplanting a population of cells enriched for human NSC, comprising the steps of contact a population containing NSC with a reagent that recognizes a determinant on a PACAP receptor, a Maxadilan receptor, or an ADCYAP1R1, selecting for cells in which there is contacted between the reagent and the determinant on the surface of the cells of the previous step to produce a population highly enriched for central nervous system stem cells; and implanting the selected cells into a non-human mammal.

Another embodiment of the invention is directed to a method of modulating a receptor for PACAP or Maxadilan on the surface of a NSC using the step of contacting the cell expressing the receptor to exogenous reagent, antibody, or affibody so that the exposure induces the NSC to proliferation, differentiation, migration or survival. In this embodiment the NSC may be derived from fetal brain, adult brain, neural cell culture or a neurosphere.

Another embodiment of the invention is directed to a method for testing an isolated candidate PACAP or Maxadilan receptor modulator compound for its ability to modulate NSC activity. In the method, the isolated compound is administered to a non-human mammal; and it is determined if the candidate compound has an effect on modulating the NSC activity in the non-human mammal. The determining step may involve comparing the neurological effects of said non-human mammal with a referenced non-human mammal not administered the candidate compound. The NSC activity may be proliferation, differentiation, migration or survival. Administration may be performed, for example by injection using any of the methods including peptide fusion or micelle delivery, discussed in this disclosure.

Another embodiment of the invention is directed to a method of increasing the amount of intracellular cAMP or intracellular adenylate cyclase activity in a NSC cell by contacting the cell with a NSC therapeutic agent.

Another embodiment of the invention is directed to a method for increasing the amount of intracellular cAMP or intracellular adenylate cyclase activity by administering PACAP or Maxadilan in a sufficient amount to a patient.

The invention also provides for pharmaceutical composition for activating intracellular adenylate cyclase activity. The composition may comprise a NSC therapeutic agent as an active ingredient In one embodiment, the NSC therapeutic agent may be pegylated. In another embodiment, the pharmaceutical composition may incorporate a growth factor. The growth factor may be EGF, VEGF or a combination thereof. The pharmaceutical composition may be in any form including, at least, a patch, a tablet, a capsule, a troche, a cachet, an elixir, an ointment, an aseptic an injectable, a molded cataplasm, a patch, a tape, a suppository or an aseptic powder. The pharmaceutical composition may have a unit dosage of between 0.1 to 2000 mg of NSC therapeutic agent. For example, the pharmaceutical composition may have a unit dosage of between 10 to 1000 mg of the NSC therapeutic agent. The pharmaceutic composition may be used for elevating intracellular cAMP or for the treatment of neurological disease.

Other features of the invention will become apparent in the course of the following description of exemplary embodiments which are given for illustration of the invention and are not intended to be limiting thereof. All references, patents and patent applications cited are hereby incorporated by reference in their entirety.

### EXAMPLES

### Example 1

### Expression of adcvaplrl Gene in Adult Mouse and Human NSC and in Neurogenic Regions of the Adult Mouse and Human Brain

### Methods

A. Mouse & Human Cultures and Mouse Neurosphere Cultures.

The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

Adult Human Neural Stem Cell (aHNSC) Cultures

A biopsy from the anterior lateral wall of the lateral ventricle was taken from an adult human patient and enzymatically dissociated in PDD (Papain 2.5 U/ml; Dispase 1 U/ml; Dnase 1 250 U/ml) in DMEM containing 4.5 mg/ml glucose and 37° C. for 20 min. The cells were gently triturated and mixed with three volumes of Human Neural Stem Cell Plating Medium (HNSCPM) (DMEM/F12; 10% fetal bovine serum (FBS)). The cells were pelleted at 250×g for 5 min. The supernatant was subsequently removed and the cells resuspended in HNSCPM, plated out on fibronectin coated culture dishes and incubated at 37° C. in 5% CO₂. The following day the expansion of the culture was initiated by change of media to aHNSC culture media (DMEM/F12; BIT 9500; EGF 20 ng/ml; FGF2 20 ng/ml). The aHNSC were split using trypsin and EDTA under standard conditions. FBS was subsequently added to inhibit the reaction and the cells collected by centrifugation at 250×g for 5 min. The aHNSC were replated in aHNSC culture media.

B. RT-PCR

Mouse Neurospheres and Lateral Ventricle Wall

The following primer pairs were designed to specifically identify the presence of adcyaplrl gene expression and its isoforms in mouse neurospheres and lateral ventricle wall tissue. Estimated band sizes for each primer pair depending on which isoform they amplify are given below:

Band size (base
pairs)

| | | |
|---|---|---|
| adcyap1r1 (hop1 form) | CCTGTCGGTGAAGGCCCTCTACACA (SEQ ID NO:1) | 801 |
| | CCCAGCCCAAGCTCAAACACAAGTC (SEQ ID NO:2) | |
| adcyap1r1 (short form) | TACTTTGATGATGCGGGATGCT (SEQ ID NO:3) | 330 |
| | AGTACAGCCACCACAAAGCCCT (SEQ ID NO:4) | |
| adcyap1r1 (hop1 form) | TACTTTGATGATGCGGGATGCT (SEQ ID NO:3) | 413 |
| | AGTACAGCCACCACAAAGCCCT (SEQ ID NO:4) | |
| adcyap1r1 (hop2 form) | TACTTTGATGATGCGGGATGCT (SEQ ID NO:3) | 410 |
| | AGTACAGCCACCACAAAGCCCT (SEQ ID NO:4) | |

Neurospheres were prepared from the LVW as stated above. 3 days after the first split, the neurospheres were harvested and total RNA isolated using Qiagen's (Hilden, Germany) RNeasy Mini Kit according to the manufacturer's instructions. Life Technology's (Gaithersburg, Md.) One-Step RT-PCR Kit was used to detect the presence of adcyaplrl mRNA. Briefly, 12.5 ng of total RNA was used in each reaction, with an annealing temperature of 55° C. To ensure that genomic contamination of the total RNA did not give rise to false positive results, an identical reaction in which the RT-taq polymerase mix was replaced by taq polymerase alone and was run in parallel with the experimental RT-PCR. The reactions were electrophoresed on a 1.5% agarose gel containing ethidium bromide and the bands visualized under UV light. Bands corresponding to the estimated length of PCR products of the desired genes were cloned into the cloning vector pGEM-Teasy and sequenced to verify their identity.

Adult Human Neural Stem Cells

The following primer pair was designed to specifically identify the presence of adcyaplrl gene expression and its isoforms in HNSC cultures. Estimated band sizes for the primer pair depending on which isoform they amplify is given below:

Band size (base
pairs)

| | | |
|---|---|---|
| adcyap1r1 (short isoform) | TACTTTGATGACACAGGCTGCT (SEQ ID NO:5) | 330 |
| | AGTACAGCCACCACAAAGCCCT (SEQ ID NO:6) | |
| adcyap1r1 (hop1/2 isoforms) | TACTTTGATGACACAGGCTGCT (SEQ ID NO:5) | 413 |
| | AGTACAGCCACCACAAAGCCCT (SEQ ID NO:6) | |

aHNSC were prepared and cultured as stated above. Total RNA isolated using Qiagen's RNeasy Mini Kit according to the manufacturer's instructions and DNase treated using Ambion DNase I and according to protocol. Life Technology's One-Step RT-PCR Kit was used to detect the presence of adcyaplrl mRNA. Briefly, 50 ng of total RNA was used in each reaction, with an annealing temperature of 55° C. To further ensure that genomic contamination of the total RNA did not give rise to false positive results, an identical reaction in which the RT-taq polymerase mix was replaced by Taq polymerase alone and was run in parallel with the experimental RT-PCR. The reactions were electrophoresed on a 1.5% agarose gel containing ethidium bromide and the bands visualised under UV light. Bands corresponding to the estimated length of PCR products of the desired genes were cloned into the cloning vector pGEM-Teasy and sequenced to verify their identity.

C. Radioactive in situ Hybridization Probes

| | | | | | |
|---|---|---|---|---|---|
| | accession | | | isoform | base pairs of |
| gene | number | name | species | specific | codi sequence |

| | | | | | |
|---|---|---|---|---|---|
| adcyap1r1 | D82935 | PAC1R | mouse | all | 1181-1475 |
| | | | | isoforms | |
| adcyap1r1 | D82935 | PAC1Rs | mouse | short | 877-1046 + |
| | | | | | 1129 |
| | | | | | 1279 |
| adcyap1r1 | D82935 | Hop1 | mouse | hop1/hop2 | 877-1279 |

Tissue Preparation and Hybridization

Sections (14 μm) of whole mouse brain and human post-mortem lateral ventricle wall and hippocampal tissue were cut on a cryostat at −17° C., thawed onto microscope slides (Superfrost Plus, BDH, UK) and fixed in 4% formaldehyde for 5 min, deproteinated for 15 min in 0.2 M HCl, treated in 0.25% acetic anhydride in 0.1 M triethanolamine buffer, pH 8.0 for 20 min and dehydrated in an ascending series of ethanol concentrations including a 5 min chloroform step prior to hybridization. To detect adcyallrl mRNA, antisense cRNA probes, both isoform specific and covering all known isoforms were transcribed from plasmids (pGEM-Teasy) containing cDNA (corresponding to bases of the coding sequence of the adcyaplrl gene shown above) and concurrently [α-³⁵S]UTP-labeled. The sections were incubated with the probe (PACR1, PACR1s, Hop1 for mouse sections; PACR1 for human sections) at 55° C. for 16 h in a hybridization buffer containing 52% formamide, 10% Dextran Sulfate, 208 mM NaCl, 2% 50×Denhardt's solution (1% Ficoll, 1% polyvinylpyrrolidene, 1% BSA) 10 mM Tris pH 8.0, 1 mM EDTA, 500 ng/ml yeast tRNA, 10 mM dithiothreitol (DTT) and 20×10⁶ cpm probe per ml buffer. After hybridization, the sections were treated with RNase A, 10 μg/ml in 0.5 M NaCl, at 37° C. for 30 min and washed in 4× saline sodium citrate (SSC; 1×SSC is 0.15 M sodium chloride, 0.015 M trisodium citrate pH 7.0) for 20 min, 2×SSC for 10 min., 1×SSC for 10 min. and 0.5×SSC for 10 min. at room temperature. A high stringency wash was carried out at 70° C. for 30 min in 0.1×SSC. All wash steps included the addition of 1 mM DTT. The sections were dehydrated in a ascending series of ethanol concentrations, dried over night and mounted in cassettes with autoradiographic films (Beta-max, Amersham) layed on top for 3 weeks. The films were developed in Kodak D-19 developer, fixed in Kodak RA-3000 diluted 1:3, rinsed and dried. The sections were then dipped in Kodak NTB-2 nuclear track emulsion diluted 1:1, exposed for six weeks, developed in Kodak D-19 for 3 min., fixed in Kodak RA-3000 fixer and counterstained with cresyl violet. The specificity of the hybridization was tested using a sense probe transcribed from the same plasmid. No hybridization signal was obtained under this condition. The emulsion dipped sections were analysed manually using a Nikon E600 microscope.

### Results

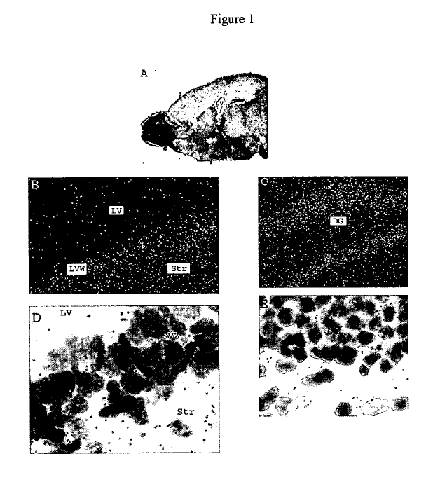

FIG. 1 represents brightfield and darkfield micrographs of adcyaplrl mRNA positive cells in coronal and sagittal sections of adult mouse brain using a probe specific for all known mouse adcyaplrl isoforms. FIG. 1A is a low magnification photomicrograph showing adcyaplrl expression in the dentate gyrus of the hippocampus and the wall of the lateral ventricle. FIGS. 1B & D shows higher magnification of the lateral ventricle wall. Note the positively labelled cells in the subventricular zone of the lateral ventricle wall. FIGS. 1C & E shows expression in the dentate gyrus of the hippocampus. Note high levels of labelling in the granular cell layer. Abbreviations: DG, dentate gyrus; GCL, granular cell layer; LV, lateral ventricle; LVW, lateral ventricle wall; Str, striatum; SVZ, subventricular zone.

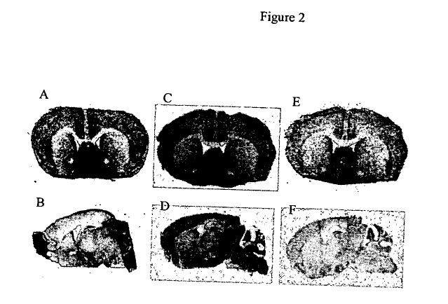

FIG. 2 shows low magnification photomicrographs of adcyaplr mRNA expression using a probe specific for all known isoforms of the gene (A,B), and probes specific for the hop1/2 isoform (C,D) and short isoform (E,F) expression in coronal (A,C,E) and sagittal (B,D,F) sections of adult mouse brain. Note the expression of both isoforms in the dentate gyrus of the hippocampus and the wall of the lateral ventricle. Abbreviations: DG, dentate gyrus; LV, lateral ventricle.

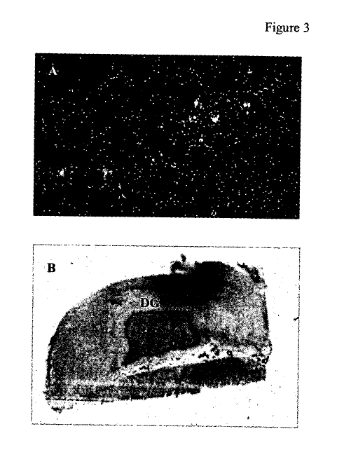

FIG. 3 shows high magnification photomicrographs of adcyaplrl mRNA positive cells in the subventricular zone of human lateral ventricle wall (A) and the human hippocampal dentate gyrus (B). Abbreviations: DG, dentate gyrus; LV, lateral ventricle.

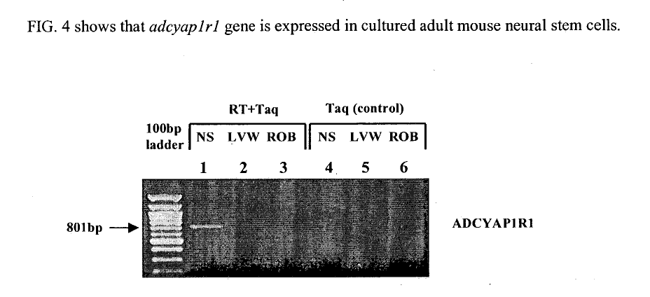

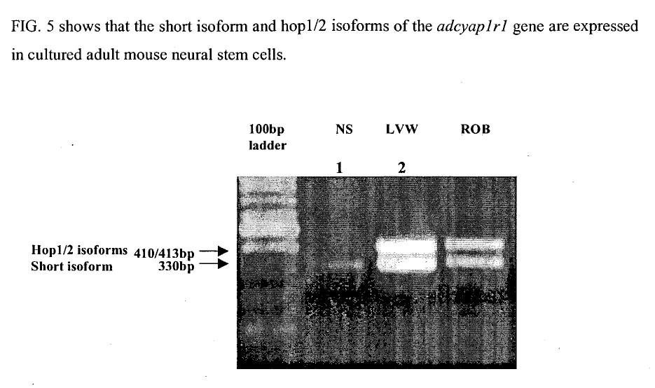

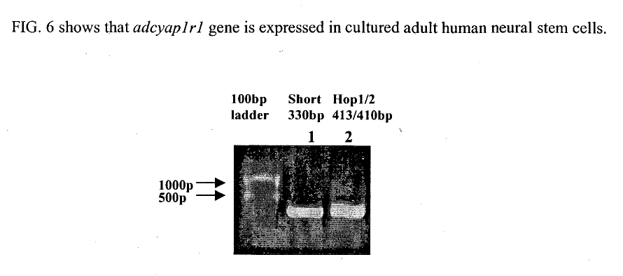

RT-PCR was performed on total RNA prepared from cultured non-adherent mouse neurospheres (NS), mouse lateral ventricle wall (LVW), rest of brain tissue (ROB) (FIG. 4 and FIG. 5) and adult HNSC (FIG. 6) using primer pairs specific for the mouse and human adcyaplrl gene. The bands indicated with an arrow correspond to the desired PCR product size of the isoform of the adcyaplrl gene they represent. FIG. 4: Mouse adeyaplrl (801 bp) ([lane1 NS; lane2 LVW; lane3 ROB]). FIG. 5: Mouse adcyaplrl short form (330 bp) and hop1/2 (413/410 bp) ([lane1 NS; lane2 LVW; lane3 ROB]). FIG. 6: Human adcyaplrl short isoform (330 bp) ([lane1]) and hop1/2 isoforms (413/410 bp) ([lane2]). Sequencing of these bands confirmed that they represent correct product indicated. In the case of the bands corresponding to the mouse hop1 and hop2, both isoforms were identified. Parallel control experiments without using any reverse transcriptase, only taq polymerase, ruled out false positive bands through genomic contamination.

### Example 2

### Adcvaplrl Stimulation by PACAP and the adcvaplrl Specific Agonist, Maxadilan, Mediates Adult Mouse NSC Proliferation in Vitro

### Methods

A. Mouse Neurosphere Cultures

The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

Chemicals for dissociation of tissue; Trypsin, Hyaluronidase and DNase were from SIGMA. Medium (DMEM 4,5 mg/ml glucose, and DMEM/F12), B27 supplement and Trypsin/EDTA were from GIBCO. All plastic ware were purchased from CorningCostar. EGF for cell cultures was from BD Biosciences.

B. Intracellular ATP Assay

Intracellular ATP levels have previously been shown to correlate to cell number (Crouch, Kozlowski et al. 1993). Mouse neurospheres, cultured as described above, from passage 2, were seeded in DMEM/F12 supplemented with B27 into a 96-well plate as single cells (10000 cells/well) to the substances to be measured were added at the concentrations indicated. After 3 days incubation, intracellular ATP was measured using the ATP-SL kit from BioThema, Sweden, according to the manufacturer's instructions. PACAP and VIP were purchased from Bachem. Maxadilan was a kind gift from Richard G Titus, Dept Microbiology, Immunology and Pathology, College of Veterinary Medicine and Biological Sciences, Colorado State University.

In experiments where signalling pathways were examined, cells were seeded as single cells as mentioned above. PACAP, 100 nM, was co incubated with 10 uM PKA inhibitor H89 (Alexis-Biochemicals) or 1 uM PLC inhibitor U73122 (Alexis-Biochemicals) or 1 uM of the PKC inhibitor Gö6976 (Sigma-Aldrich). Cells were incubated for 4 days before measurements of ATP.

In the experiment analysing the combinatory effect of PACAP and EGF, PACAP at 100 nM was co-incubated with 3 nM EGF for 3 days.

C. Thymidine Incorporation

To determine thymidine incorporation into DNA, neurospheres were split and seeded in Neurosphere medium as single cells in 96-well plates, 10,000 cells/well. Substances to be measured were added in quadruplicates and cells were incubated at 37° C. for 3 days. 3H-thymidine, 10 uCi/ml, was present the last 24 hours. Cells were harvested on to a filter paper and radioactivity was measured. 3H-thymidine (6,7 Ci/mmol) was from PerkinElmer.

D. Culture of NSC for Cell Counting

Anterior lateral ventricle wall was dissected and the cells treated as stated above, however, in place of EGF, the medium was supplemented either with PACAP (100 nM), VEGF (1 nM) or a combination of both at the stated concentrations. The cell suspension was plated into wells of a 24 well plate. The medium was further supplemented with PACAP and/or VEGF every 2 days. After 7 days in culture, the NSC washed in phosphate buffered saline and dissociated with trypsin/EDTA. Cells numbers per well were counted using a Bürker chamber. VEGF was purchased from R&D Systems.

E. Western Blot

Adult mouse NSC and aHNSC were prepared and cultured as stated in Example 2 (Method A) and Example 1 (Method A). Cultures were exposed to either 10 nM PACAP or EGF (1 nM) and FGF (1 nM) for the times indicated. After treatment, the cells were lysed in lysis buffer, as shown in Patrone et al. (Patrone, Andersson et al. 1999). DNA content was detemined by the use of a Pico Green-kit for ds DNA quantitation of cell extracts. Total protein measurements were performed with Nano Orange-kit. Equal amounts of protein were run on a gradient gel 4-12% Bis-Tris gel (NuPage/Mops buffer) and transferred to a nitrocellulose membrane. Western blot was performed and phosphorylated CREB was labelled using a rabbit anti phospho-CREB (1:1000, Upstate biotechnology), a secondary anti rabbit HRP antibodies (1:10.000). Phosphorylated CREB protein bands (43 kD) were detected using the ECL-kit (Amersham).

F. AP-1 Transcription Factor Reporter Assay

Vectors: Vector DNA containing reporter elements for AP-1 and empty cloning vector (pTAL), with a luciferace reporter were purchased from Promega. The vectors were propagated in E. coli strain JM-109 (Promega) and purified with Qiagens maxi-prep Kit (Qiagen). The concentration was diluted to approximately 1 mg/ml per vector.

Transfection: Transient transfection was performed by seeding cells as above in suspension neurosphere cell culture and adherent neurosphere cell culture (1 to 3 day incubation) using 30,000 single cells/well. Each well was transiently transfected with 0.1 mg plasmid, 0.6 μg Nupherin-neuron (Biomol) and 0,6 μl of Fugene-6 (Roche). Plasmid and Nupherin was mixed and diluted in DMEM/F12 to a total volume of 50 μl/well, and were incubated for 15 minutes prior to mixing with Fugene-6 that were diluted in DMEM/F12 to a total volume of 50 μl/well. The mix of plasmid and Nupherin and Fugene-6 were incubated for 20 min. The volume was adjusted to 100 μl prior to addition of 100 μl/well of transfection reagent.

Assay: The following day the transiently transfected cells were applied with PACAP (Bachem) to final concentration of 100 nM/well, in the presence or absence of MEK inhibitor, PD89059 (Sigma-Aldrich). Luciferace activity was analysed with steady glow (Promega) according to the instructions of the manufacturer. Cells were analysed 20 hours after induction with PACAP.

### Results

PACAP Stimulates Adult Mouse NSC Proliferation in Non-Adherent Culture Conditions

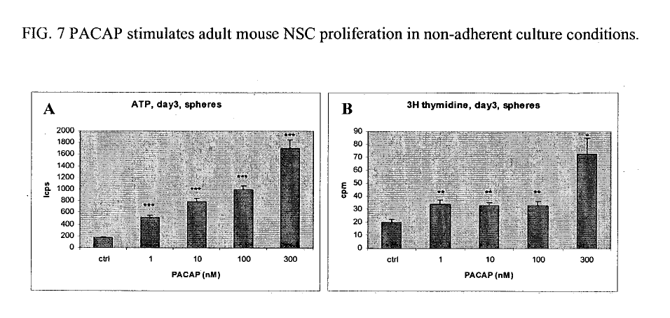

To determine the effect of PACAP on neural stem cells in culture, mouse adult neural stem cells derived from the lateral ventricle wall of the brain, expanded in EGF as neurospheres followed by enzymatic dissociation using trypsin, were cultured in Neurosphere medium supplemented with varying concentrations of PACAP, under non-adherent conditions, for 3 days. To ascertain whether there was an increase in cell number of PACAP treated cells relative to control cells, an assay measuring intracellular ATP levels, shown previously to correlate with cell number (Crouch, Kozlowski et al. 1993), was employed. FIG. 7A shows a statistically significant increase in intracellular ATP levels, and hence cell number, in response to PACAP in a dose-dependent manner. To ascertain whether the effect of PACAP is through proliferation, incorporation of tritiated thymidine was used to assess DNA synthesis in NSC. Greater incorporation of tritiated thymidine was observed with all PACAP treatments relative to controls, indicating that PACAP is eliciting a proliferating response in NSC under non-adherent conditions (FIG. 7B). Data shown in FIG. 7 are from experiments performed in sextuplicate. Bars represent ±SEM. Levels of significance of increases above control were determined by a paired Student t test; * P<0.05, ** P<0.01, *** P<0.005.

Maxadilan Stimulates Adult Mouse NSC Proliferation in Non-Adherent Culture Conditions

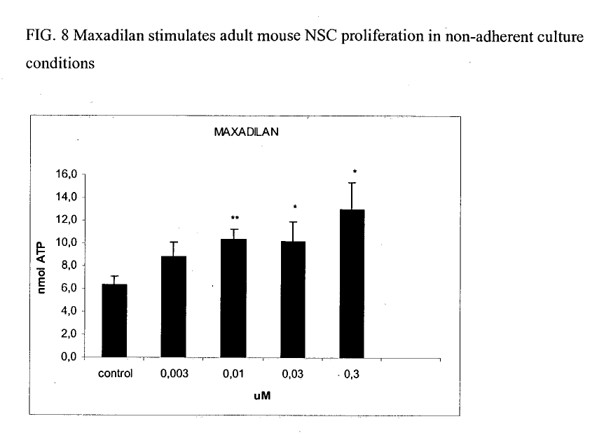

Maxadilan binds specifically to only ADCYAP1R1 (Moro and Lerner 1997). To determine the effect of Maxadilan and sMax on neural stem cells in culture, mouse adult neural stem cells derived from the lateral ventricle wall of the brain, expanded in EGF as neurospheres followed by enzymatic dissociation using trypsin, were cultured in Neurosphere medium supplemented with varying concentrations of Maxadilan under non-adherent conditions, for 3 days. To ascertain whether there was an increase in cell number of Maxadilan treated cells relative to control cells, an assay measuring intracellular ATP levels. FIG. 8 shows a statistically significant increase in intracellular ATP levels, and hence cell number, in response to Maxadilan in a dose-dependent manner. This data indicates that the effects of Maxadilan, mediated through ADCYAP1R1, can elicit NSC proliferation. Data shown in FIG. 8 are from experiments performed in sextuplicate. Bars represent ±SEM. Levels of significance of increases above control were determined by a paired Student t test; *P<0.0, **P<0.005.

PACAP and EGF Synergistically Proliferate Adult Mouse NSC in vitro

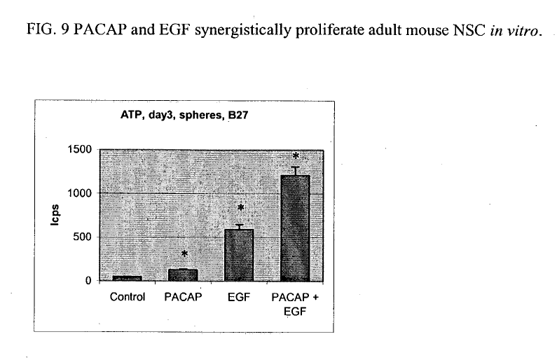

NSC were treated with PACAP, EGF or a combination of the two. While either of the two factors alone increased intracellular ATP levels, and hence NSC number, when combined, there was a further elevation of the values of which are indicative of a synergistic effect between the two factors (FIG. 9). Data shown are from experiments performed in octuplicate. Bars represent ±SEM. Levels of significance of increases above control were determined by a paired Student t test; * P<0.005.

PACAP and VEGF Have an Additive Effect on Adult Mouse NSC Number in vitro

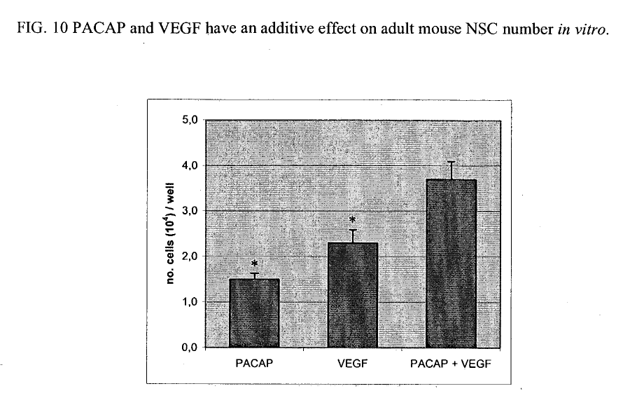

NSC from LVW were prepared as described in Example 2 (Method A), however EGF was either omitted or replaced with PACAP (100 nM), VEGF (1 nM), or PACAP (100 nM) plus VEGF (1 nM). NSC were cultured for 7 days after which time the cells were dissociated with trypsin and counted. A significant increase in cell number is observed on treatment with VEGF relative to PACAP treated NSC. However, PACAP in combination with VEGF has greater effect on cell number (FIG. 10), the values of which are indicative of an additive effect between these two factors. Data shown are from experiments performed in quadruplicate. Bars represent ±SEM. Levels of significance relative to PACAP +VEGF were determined by a paired Student t test; * P<0.05.

PACAP Stimulated Adult Mouse NSC Proliferation is Inhibited by PLC and PKC Inhibitors but not PKA Inhibition.

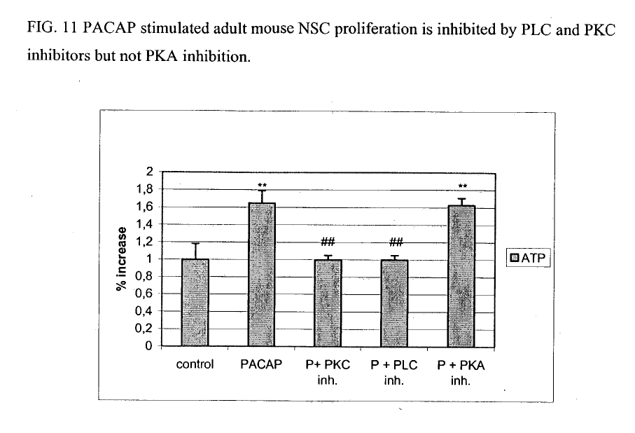

Intracellular pathways triggered by the effects of PACAP through ADCYAP1R1 have been well studied. ADCYAP1R1, upon PACAP stimulation, can elicit both PLC/PKC and PKA cascades. To determine the relative importance of these cascades in the proliferating effect of PACAP on NSC cultures, inhibitors of PLC (U73122), PKC (Gö6976) and PKA (H89) were applied to PACAP treated NSC and assayed for intracellular ATP. FIG. 11 shows the PACAP significantly increases intracellular ATP levels relative to control, an effect that is unaltered by the presence of the PKA inhibitor. In contrast, both the PLC and PKC inhibitors entirely negate the effect of PACAP. This data illustrates that the PLC/PKC cascade plays a significant role in mediating the effects of PACAP. Bars represent ±SEM. Levels of significance of increases above control were determined by a paired Student t test; ** P<0.01. Levels of significance of change relative to PACAP treatement were determined by a paired Student t test; ##P<0.01.

PACAP Stimulates CREB Phosphorylation in Adult Mouse and Adult Human NSC

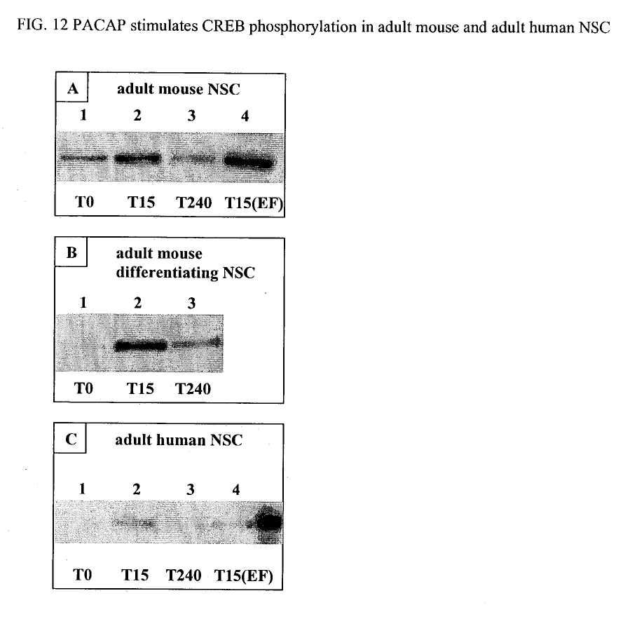

The effects of PACAP, through ADCYAP1R1 stimulation, have previously been shown to trigger phosphorylation of the transcription factor CREB. To determine whether adult mouse NSC grown under non-adherent culture conditions and undifferentiated aHNSC evoke CREB phosphorylation upon PACAP treatment, PACAP was applied to the above cells for 15 min and 240 min and analyzed using Western blotting techniques with an antibody specific for the phosphorylated form of CREB. FIG. 12A shows that CREB phosphorylation is upregulated in adult mouse NSC upon 15 min exposure to PACAP, while after 240 min CREB phosphorylation return to basal levels. This response to PACAP was essentially the same for undifferentiated aHNSC, with an initial strong upregulation of CREB phosphorylation after 15 min, and a subsequent lowering of phosphorylation levels after 240 min to slightly above basal levels (FIG. 12C).

PACAP Stimulates AP-1 Transcription Through the MEK Signaling Pathway

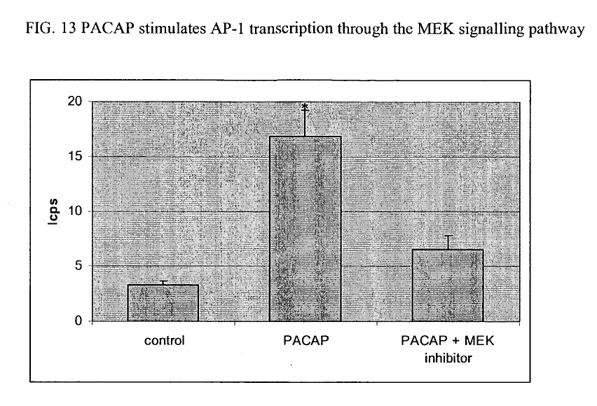

To understand whether PACAP induces the expression of the transcription factor AP-1 (composed of c-Fos and c-Jun) in adult mouse NSC grown under non-adherent culture conditions, adult mouse NSC were transfected with an AP-1-luciferace reporter vector and PACAP (100 nM) applied. FIG. 13 shows that AP-1 expression is significantly induced by PACAP, a response that can be inhibited by the MEK inhibitor, PD89059 (10 μM). The luciferace activity was compared to non-induced cells (control). Data shown are from experiments performed in octuplicate. Bars represent ±SEM. Levels of significance of increases above control were determined by a paired Student t test; * P<0.05.

### Example 3

### VIP Stimulates Adult Mouse NSC Proliferation in Vitro

### Methods

See Example 2 (Method A & B).

### Results

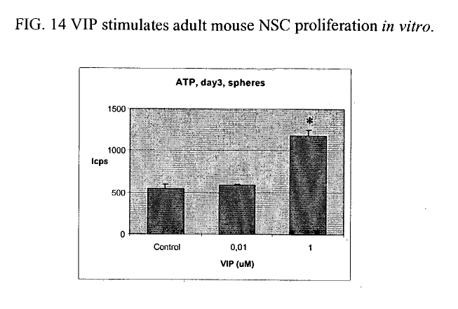

To determine the effect of VIP on neural stem cells in culture, mouse adult neural stem cells derived from the lateral ventricle wall of the brain, expanded in EGF as neurospheres followed by enzymatic dissociation using trypsin, were cultured in Neurosphere medium supplemented with varying concentrations of VIP, under non-adherent conditions, for 3 days. To ascertain whether there was an increase in cell number of VIP treated cells relative to control cells, an assay measuring intracellular ATP levels, shown previously to correlate with cell number (Crouch, Kozlowski et al. 1993), was employed. FIG. 14 shows a statistically significant increase in intracellular ATP, and hence cell number, in response to VIP at 1 μM concentration. Data shown are from experiments performed in sextuplicate. Bars represent ±SEM. Levels of significance of increases above control were determined by a paired Student t test; * P<0.005.

### Example 4

### PACAP Stimulates Primary Adult Mouse NSC to Proliferate, While Retaining the Self-Renewal and Multi-Potentiality Characteristics of Neural Stem Cells

### Methods

A. Culture of Neurospheres for Counting and Photographing

Anterior lateral wall of the lateral ventricle was dissociated as described in Example 2 (Method A), and the cells resuspended in Neurosphere medium without EGF. The cell suspension was divided into a 24-well plate with triplicates of control (no addition), PACAP treated and EGF (1 nM) treated cells. The final concentration of PACAP was 100 nM. After 7 days the spheres were counted and photographed using a Nikon Eclipse TE300 microscope and Nikon Spot Insight camera.

B. Self Renewal and Multipotency Assay

Anterior lateral wall of the lateral ventricle was dissociated as described in Example 2 (Method A), and the cells resuspended in Neurosphere medium without EGF. The cell suspension was divided into a 24-well plate in triplicate and PACAP added to a final concentration of 100 nM. After 7 days, the neurospheres were split into single cells, as described in Example 1 (Method A), but in the absence of EGF. The cells were replated as single cells, PACAP added to 100 nM and incubated a further 7 days. The cells were passaged one further time, and plated as single cells in Neurosphere medium supplemented with 1% Fetal Calf Serum (Gibco) and 100 nM PACAP (Bachem) onto poly-D-lysine plates. The cells were incubated over-night in which time they adhered to the poly-D-lysine plates and the medium was changed to Neurosphere medium supplemented with 100 nM PACAP38, but absent of Fetal Calf Serum. The cells were cultured for a further 3 days, after which they were washed twice in PBS (Gibco) and fixed for 15 min at room temperature with 4% Formaldehyde (Sigma) and permeabilised for 20 minutes at room temperature in 0,1% Triton X-100 (Sigma) in PBS. After fixation and permeabilisation the cells were labelled with mouse monoclonal anti β-III Tubulin (1:1000 Promega), rabbit anti GFAP (1:500 Sigma), and mouse anti CNPase (1:500 Sigma). Primary antibodies were visualized with anti mouse Texas-Red and anti rabbit FITC (1:500 Vector Laboratories). All antibodies were diluted in PBS with 0,1% Triton X-l00.

### Results

PACAP Stimulates Primary Adult Mouse NSC Proliferation, Neurosphere Formation and Self-Renewal

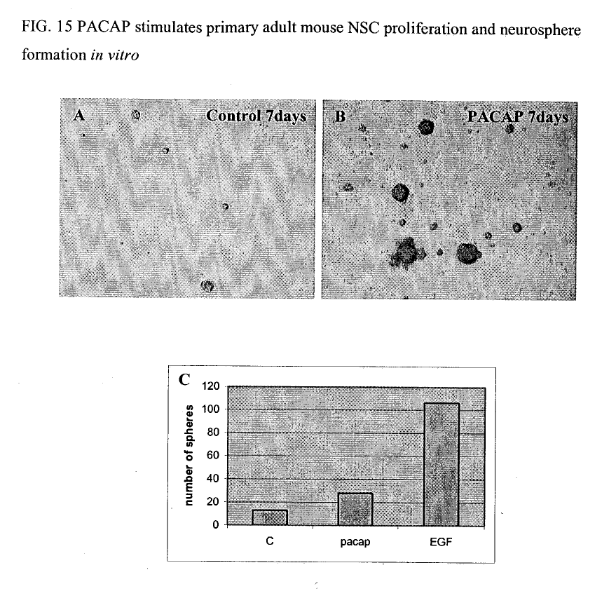

To determine whether PACAP, in the absence of mitogens, can alone act on primary adult NSC to stimulate their proliferation, anterior lateral wall of the lateral ventricle was dissociated and the cells resuspended in Neurosphere medium in which EGF was either omitted or replaced with PACAP (100 nM). NSC were cultured for 7 days after which time the NSC were inspected for growth and morphology. FIG. 15 shows NSC treated with PACAP growing in a neurosphere formation, the sizes (A & B) and number (C) of which are observably greater than that of the control. PACAP-treated neurospheres retained their ability to self-renew, generating secondary and teriatary neurospheres in the presence of PACAP and absence of other mitogens. Multiply-passaged PACAP treated neurospheres showed no relevant differences compared to EGF-treated neurospheres.

Adult Mouse NSC Proliferated by PACAP Treatment Retain Their Multipotenial

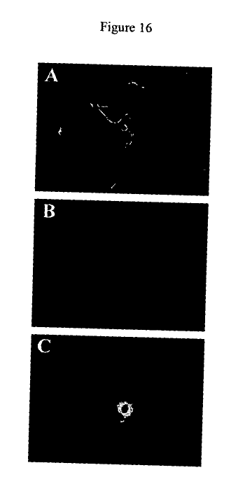

To investigate if PACAP-treated NSC retain the potential to differentiate into the three cell lineages of the brain, namely, neurons, astrocytes and oligodendrocytes, neurospheres were split and the cells allowed to spontaneously differentiate on a poly-D-lysine precoated petri dishes. The results shown in FIG. 16 show cells were immunoreactive against the neuronal marker tubulin (A), the astrocyte marker GFAP (B) and oligodendrocyte marker CNPase (C), thus retaining the potential of generating all the cells of the CNS.

### Example 5

### PACAP Promotes in Vitro Survival of Cells Derived From Adult Mouse NSC

### Methods

A. Cell Culture

Adult mouse NSC were cultivated from the anterior lateral wall of the lateral ventricle of 5-6 week old mice as described in Example 2 (Method A).

B. Intracellular ATP Assay

Intracellular ATP levels have previously been shown to correlate to cell number (Crouch, Kozlowski et al. 1993). Mouse neurospheres, cultured as described above, from passage 2, were seeded in DMEM/F12 supplemented with B27 and 1% Fetal Calf Serum into a 96-well plate coated with poly-D-lysine as single cells (30000 cells/well) and cultured over-night. The following day the medium was replaced with Neurosphere medium and PACAP added to the concentrations indicated. After 3 days incubation, intracellular ATP was measured using the ATP-SL kit from BioThema, Sweden, according to the manufacturer's instructions.

For photographic purposes, a protocol identical to the above was used except that cells were plated in 24-well plates, but at the same density. After 7 days the cells were photographed using a Nikon Eclipse TE300 microscope and Nikon Spot Insight camera.

C. Thymidine Incorporation

To determine thymidine incorporation into DNA, mouse neurospheres, cultured as described above, from passage 2, were seeded in DMEM/F12 supplemented with B27 and 1% Fetal Calf Serum into a 96-well plate coated with poly-D-lysine as single cells (30000 cells/well) and cultured over-night. The following day the medium was replaced with Neurosphere medium and PACAP added to the concentrations indicated for a further 3 days. 3H-thymidine, 10 uCi/ml, was present the last 24 hours. Cells were harvested on to a filter paper and radioactivity was measured. 3H-thymidine (6,7 Ci/mmol) was from PerkinElmer.

E. Western Blot

Adult mouse NSC and aHNSC were prepared and cultured as stated in Example 5 (Method A & B) and Example 2 (Method A), respectively. Cultures were exposed to 10 nM PACAP for the times indicated. After treatment, the cells were lysed in lysis buffer, as shown in Patrone et al. (Patrone, Andersson et al. 1999). DNA content was detemined by the use of a Pico Green-kit for ds DNA quantitation of cell extracts. Total protein measurements were performed with Nano Orange-kit. Equal amounts of protein were run on a gradient gel 4-12% Bis-Tris gel (NuPage/Mops buffer) and transferred to a nitrocellulose membrane. Western blot was performed and phosphorylated CREB was labelled using a rabbit anti phospho-CREB (1:1000, Upstate biotechnology), a secondary anti rabbit HRP antibodies (1:10.000). Phosphorylated CREB protein bands (43 kD) were detected using the ECL-kit (Amersham).

### Results

PACAP Promotes in vitro Survival of Cells Derived From Adult Mouse NSC

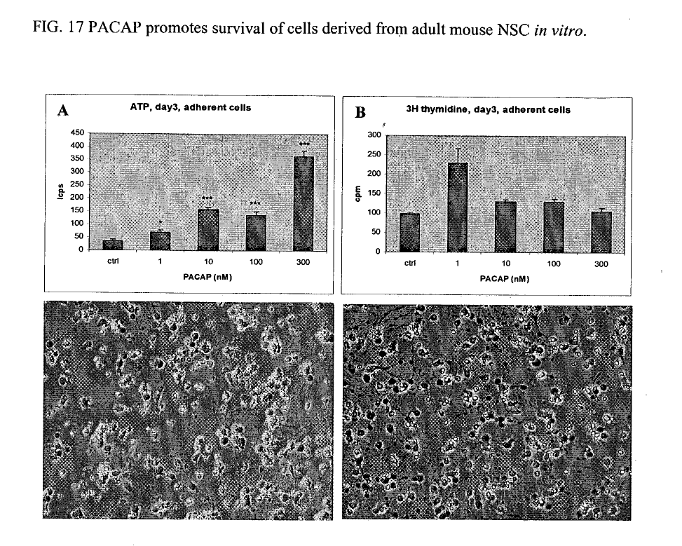

To determine the effect of PACAP on differentiating neural stem cells in culture, mouse adult neural stem cells derived from the lateral ventricle wall of the brain, expanded in EGF as neurospheres followed by enzymatic dissociation using trypsin, were cultured in Neurosphere medium supplemented with varying concentrations of PACAP, under adherent conditions, for 3 days. To ascertain whether there was an increase in cell number of PACAP treated cells relative to control cells, an assay measuring intracellular ATP levels, shown previously to correlate with cell number (Crouch, Kozlowski et al. 1993), was employed. FIG. 17A shows a statistically significant increase in intracellular ATP levels, and hence cell number, in response to PACAP in a dose-dependent manner. To ascertain whether the effect of PACAP is through proliferation, incorporation of tritiated thymidine was used to assess DNA synthesis. No significant difference in incorporation of tritiated thymidine was observed any PACAP treatments relative to controls, indicating PACAP is eliciting a survival response in the differentiating NSC (FIG. 17B). Phase contrast images of the adherently grown NSC untreated and treated with 100 nM PACAP for 96 hours are shown in FIGS. C and D, respectively. Data shown in FIGS. 17A & B are from experiments performed in sextuplicate. Bars represent ±SEM. Levels of significance of increases above control were determined by a paired Student t test; * P<0.05, *** P<0.005.

PACAP Stimulates CREB Phosphorylation in Differentiating Adult Mouse NSC

The effects of PACAP, through ADCYAP1R1 stimulation, have previously been shown to trigger phosphorylation of the transcription factor CREB. To determine whether differentiating adult mouse NSC grown under adherent culture conditions evoke CREB phosphorylation upon PACAP treatment, PACAP was applied to the above cells for 15 min and 240 min and analysed using Western blotting techniques with an antibody specific for the phosphorylated form of CREB. FIG. 12B shows that CREB phosphorylation is upregulated in differentiating adult mouse NSC upon 15 min exposure to PACAP, while after 240 min CREB phosphorylation is slightly above basal levels.

### Example 6

### PACAP Promotes Adult Mouse NSC proliferation and Neurogenesis in Vivo

### Methods

A. Implantation of Osmotic Pumps and PACAP/BrdU Infusion

10 week old male mice (C57B1/2), maintained on a 12 hr light/dark cycle with food and water ad libidum, were infused in the right lateral ventricle with PACAP38 (Bachem), using a Alzet pump (1007D), for 3,5 days or 7 days at a daily dose of 31 ng/day (600 nM PACAP pump concentration infused at a rate of 0.5 μl/hr). Bromodeoxyuridine (BrdU) (50 mg/ml) was also included in the infusion vehicle (0.9% saline containing 1 mg/ml mouse serum albumin (Sigma)) to enable measurement of proliferation by quantitation of BrdU incorporation in the DNA. The group of infused with either PACAP/BrdU or vehicle/BrdU for 3,5 days and sacrificed at this time point. The group infused with either PACAP/BrdU or vehicle/BrdU for 7 days were allowed to survive a further 10 days. After the allotted time the mice were sacrificed, perfused with PBS the brains removed and frozen at −70° C. prior to sectioning for immumohistochemical analysis.

B. Immunohistochemistry

Brains were cut into 14-μm coronal sections using a cryostat-microtome. The sections were thawed onto pretreated slides and fixed in 4% (wt/vol) paraformaldehyde/PBS for 10 min. After washing in PBS, the sections were treated with 2M HCl at 37° C. for 30 min to increase accessibility of the anti-BrdU antibody to the cell nuclei. The sections were rinsed in PBS and transferred to blocking solution (PBS; 0.1% Tween; 10% goat serum) overnight at 4° C. Primary antibody (rat anti-BrdU, Harlan Sera Labs) was applied at 1:100 in blocking solution for 90 min at room temperature. After washing in PBS/0.1% Tween for 3×30 min, secondary biotinylated antibody (goat anti-rat, VectorLabs) was added at a 1:200 dilution in blocking solution for 60 min at room temperature. The sections were washed for 2 hours prior to treatment with Vectastain Kit (VectorLabs) according to the manufacturer's protocol. After 1 hour of washing, the BrdU-antibody complex was detected using 0.05% diaminobenzidine with 0.01% H₂O₂, and counterstained with Hematoxylin. The sections were dehydrated in a graded series of ethanol concentrations, followed by xylene and 99% ethanol, and mounted in Pertex. Sections were visualised using a Nikon Eclipse E600 microscope and pictures taken with a Spot Insight CCD camera.

The procedure for doubling labeling of BrdU with NeuN was performed sequentially. Briefly, following fixation in 4% (wt/vol) paraformaldehyde/PBS for 10 min, the sections were incubated in overnight blocking solution (PBS; 0.1% Tween; 10% horse serum) at 4° C. Anti-NeuN (mouse, Chemicon) was applied at 1:100 in blocking solution for 60 min at room temperature. After washing in PBS/0.1% Tween for 3×30 min, secondary FITC-conjugated antibody (horse anti-mouse, Vector Laboratories, CA) was added at a 1:200 dilution in their appropriate blocking solution for 60 min at room temperature. The sections were washed for 2 hours prior to post-fixation in 4% (wt/vol) paraformaldehyde/PBS for 10 min followed by treatment with 2M HCl at 37° C. for 30 min to increase accessibility of the anti-BrdU antibody to the cell nuclei. The sections were rinsed in PBS and transferred to blocking solution/(PBS; 0.1% Tween; 10% goat serum) overnight at 4° C. Primary antibody (rat anti-BrdU, Harlan Sera Labs) was applied at 1:100 in blocking solution for 90 min at room temperature. After washing in PBS/0.1% Tween for 3×30 min, secondary Texas Red-conjugated antibody (goat anti-rat, VectorLabs) was added at a 1:200 dilution in blocking solution for 60 min at room temperature. The sections were washed for 2 hours prior to mounting onto glass slides. Sections were visualised using a Nikon Eclipse E600 microscope and pictures taken with a Spot Insight CCD camera.

C. Quantification and Statsistical Analysis

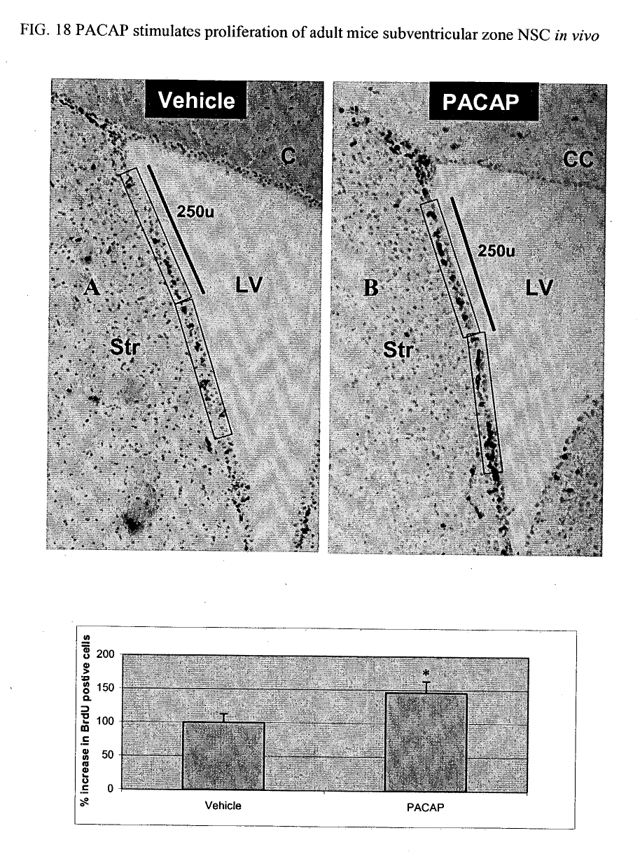

For all BrdU labelling experiments, three to six sections per animal were analysed. For the hippocampus, sections divided between the anterior, middle and posterior portions of the dorsal hippocampus were analysed in an area encompassing the entire granule cell layer (superior and inferior blades) including the sub-granular zone which was defined as extending a maximum of two cell widths into the hilus region. Based on anatomical landmarks, equivalent sections from control and experimental animals were chosen and coded by one of the authors, and remained concealed to the examiner throughout the study. The number of BrdU-labelled cells per area of dentate granule cell layer was counted manually. For the lateral ventricle wall, sections were collected posterior to the genus of corpus callosum and anterior to the closing of anterior commisura, corresponding to coronal plates number 22-29 of the Atlas over the mouse brain (Paxinos, G & Franklin, KBJ. The Mouse Brain in Stereotaxic Coordinates, Second Edition). As above, equivalent sections from control and experimental animals were chosen and coded by one of the authors, and remained concealed to the examiner throughout the study. BrdU-positive cells were counted along 2×250 micrometer strips of the lateral ventricle wall (see FIG. 18). Area measurements of both the dentate granule cell layer and the “counted” region of the lateral ventricle wall (including the ependymal cell layer and subventricular zone) were made from each slide used for the cell counts. The experimental group mean value was compared with the control group mean value. Results of the dentate gyrus BrdU counts are expressed as the average number of BrdU-positive cells per area (mm²) for each individual animal and reported as the mean ±SEM. Data generated analyzing lateral ventricle wall sections come from two independent experiments. The numbers of BrdU-positive cells per area (mm²) are expressed as percentage of the mean ±SEM of the control group. Differences between means were determined by Student's t test.

For all BrdU/NeuN co-labelling experiments, one to two sections per animal were analysed. For the hippocampus, sections were analysed in an area encompassing the entire granule cell layer (superior and inferior blades) including the sub-granular zone which was defined as extending a maximum of two cell widths into the hilus region. Based on anatomical landmarks, equivalent sections from control and experimental animals were chosen and coded by one of the authors, and remained concealed to the examiner throughout the study. The number of BrdU/NeuN-labelled cells per area of dentate granule cell layer was counted manually. Area measurements of the dentate granule cell layer were made from each slide used for the cell counts. The experimental group mean value was compared with the control group mean value. Results of the dentate gyrus BrdU/NeuN counts are expressed as the average number of BrdU/NeuN-positive cells per dentate gyrus for each individual animal and reported as the mean ±SEM.

### Results

PACAP Stimulates Proliferation of Adult Mice Subventricular Zone NSC in vivo

PACAP (600 nM pump concentration) or vehicle, and BrdU (50 mg/ml pump concentration) was infused into the lateral ventricle of adult male mice at a rate of 0.5 μl/hr for 3.5 days. BrdU incorporation into the nuclei of dividing cells is a standard method of labeling proliferating populations of cells, and has become a well used marker for dividing stem cells and their progeny (Zhang, Zhang et al. 2001). BrdU incorporated into the nuclei of proliferating cells was detected by DAB-immunohistochemistry, and all of the nuclei were counterstained with hematoxylin. Coronal sections through the lateral ventricle show BrdU labeling of a substantially greater number of cells in PACAP treated mice (FIG. 18B) relative to mice treated with vehicle (FIG. 18A). Data are representative fields from two independent experiments. For quantification (FIG. 18C), 3-6 sections were taken for each animal and counted manually in the regions boxed. Immunoreactive cells for BrdU were counted and expressed as a mean percentage ±SEM (n=10) of BrdU-immunopositive vehicle treated animals (n=11) (*p<0.05 relative to Vehicle). CC, corpus callosum; LV, lateral ventricle; Str, striatum. The quantitative data show a significant increase in proliferation in the subventricular zone of the lateral ventricle wall upon PACAP treatment relative to vehicle.

PACAP Stimulates Proliferation of Adult Mice Hippocampal NSC/Neural Progenitors Cells in vivo

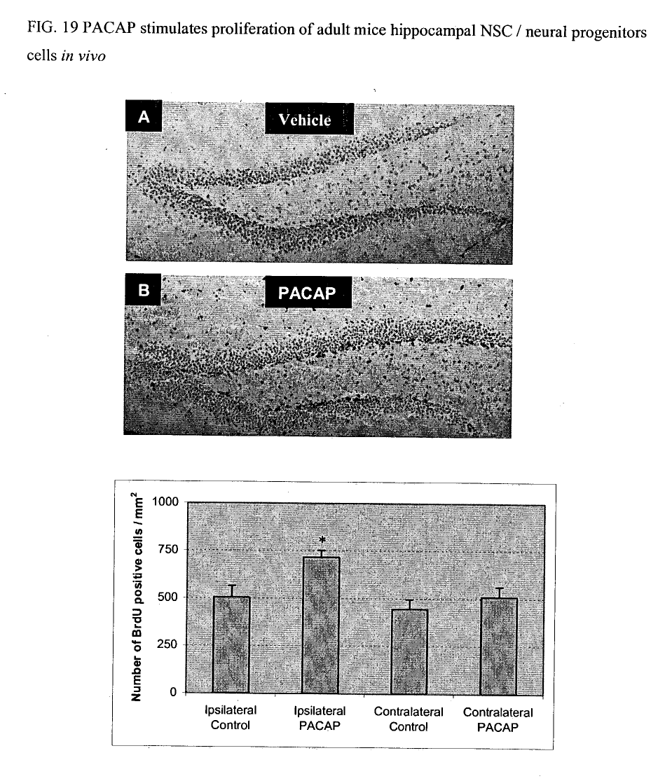

PACAP (600 nM pump concentration) or vehicle, and BrdU (50 mg/ml pump concentration) was infused into the lateral ventricle of adult male mice at a rate of 0.5 μl/hr for 3.5 days. BrdU incorporation into the nuclei of dividing cells is a standard method of labeling proliferating populations of cells, and has become a well used marker for dividing stem cells and their progeny (Zhang, Zhang et al. 2001). BrdU incorporated into the nuclei of proliferating cells was detected by DAB-immunohistochemistry, and all of the nuclei were counterstained with hematoxylin. Coronal sections through the ipsilateral dentate gyrus show BrdU labeling of a substantially greater number of cells in PACAP treated mice (FIG. 19B) relative to mice treated with vehicle (FIG. 19A). For quantification (FIG. 19C), sections were taken in triplicate for each animal and counted manually, both ipsi and contralaterally, in an area encompassing the entire granule cell layer (superior and inferior blades) including the sub-granular zone which was defined as extending a maximum of two cell widths into the hilus region. Results are expressed as the mean ±SEM number of BrdU positive cells/mm² (PACAP n=7) (Vehicle n=8) (**p <0.005 relative to Vehicle). The quantitative data show a significant increase in proliferation the ipsilateral dentate gyrus upon PACAP treatment relative to vehicle.

PACAP Stimulates in vivo Hippocampal Neurogenesis in Adult Mice

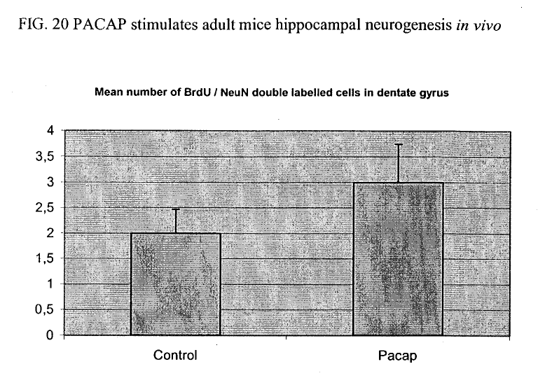

PACAP (600 nM pump concentration) or vehicle, and BrdU (50 mg/ml pump concentration) was infused into the lateral ventricle of adult male mice at a rate of 0.5 μl/hr for 7 days and allowed to survive a further 10 days before sacrifice. BrdU incorporated into the nuclei of proliferating cells and NeuN staining was detected by fluorescent-immunohistochemistry and visualised using a BioRad Radiance Confocal microscope. Double-labelled cells were verified in 12 focal planes over the neuron. For quantification (FIG. 20), sections were taken for each animal and counted manually, ipsilaterally, in an area encompassing the entire granule cell layer (superior and inferior blades) including the sub-granular zone which was defined as extending a maximum of two cell widths into the hilus region. Results are expressed as the mean ±SEM number of BrdU/NeuN positive cells/dentate gyrus (PACAP n=6) (Vehicle n=8). The quantitative data show an increase in neurogenesis in the ipsilateral dentate gyrus upon PACAP treatment relative to vehicle.

### Example 7

### Biopolymer Sequences

The DNA and protein sequences referenced in this patent are as listed below.

A. PACAP (ADCYAP1) Locus Link ID: 116 (Human); 11516 (Mouse)

| GenBank | |
|---|---|
| Accession | |
| Number | Description |
| NM_001117 | Homo sapiens adenylate cyclase activating polypeptide |
| | 1 (pituitary) (ADCYAP1), mRNA |
| NM_009625 | Mus musculus adenylate cyclase activating polypeptide |
| | 1 (pituitary) (Adcyap1) |
| NP_001108 | Homo sapiens adenylate cyclase activating polypeptide |
| | precursor (ADCYAP1) |
| NP_033755 | Mus musculus adenylate cyclase activating polypeptide |
| | precursor (Adcyap1) |

B. VIP Locus Link ID: 7432 (Human); 22353 (Mouse)

| GenBank | |
|---|---|
| Accession Number | Description |
| NM_003381 | Homo sapiens vasoactive intestinal |
| | peptide (VIP), mRNA |
| XM_125478 | Mus musculus vasoactive intestinal |
| | peptide (Vip), mRNA |
| NP_003372 | Homo sapiens vasoactive intestinal |
| | peptide (VIP) |
| XP_125478 | Mus musculus vasoactive intestinal |
| | peptide (Vip) |

C. ADCYAP1R1 Locus Link ID: 117 (Human); 11516 (Mouse)

| GenBank | |
|---|---|
| Accession | |
| Number | Description |
| NM_001118 | Homo sapiens adenylate cyclase activating polypeptide |
| | 1 (pituitary) receptor type I (ADCYAP1R1), mRNA |
| NM_007407 | Mus musculus adenylate cyclase activating polypeptide |
| | 1 (pituitary) receptor type I (Adcyap1r1), mRNA |
| NP_001109 | Homo sapiens type I adenylate cyclase activating |
| | polypeptide receptor precursor; adenylate cyclase |
| | activating polypeptide 1 (pituitary) receptor type 1 |
| | (ADCYAP1R1) |
| NP_031433 | Mus musculus type I adenylate cyclase activating |
| | polypeptide receptor precursor; adenylate cyclase |
| | activating polypeptide 1 (pituitary) receptor type 1 |
| | (Adcyap1r1) |

Splice Variants of ADCYAP1R1 (Human)

Pantaloni, C., Brabet, P., Bilanges, B., Dumuis, A., Houssami, S., Spengler, D., Bockaert, J. and Journot, L.

Alternative splicing in the N-terminal extracellular domain of the pituitary adenylate cyclase-activating polypeptide (PACAP) receptor modulates receptor selectivity and relative potencies of PACAP-27 and PACAP-38 in phospholipase C activation

J. Biol. Chem. 271 (36), 22146-22151 (1996)

MEDLINE 96355616

PUBMED 8703026

Pisegna, J. R. and Wank, S. A.

Cloning and characterization of the signal transduction of four splice variants of the human pituitary adenylate cyclase activating polypeptide receptor. Evidence for dual coupling to adenylate cyclase and phospholipase C

J. Biol. Chem. 271 (29), 17267-17274 (1996)

MEDLINE 96291878

PUBMED 8663363

Short ADCYAP1R1 Isoform

(SEQ ID NO:7)

ATGGCTGGTG TCGTGCACGT TTCCCTGGCT GCTCACTGCG
GGGCCTGTCC GTGGGGCCGG GGCAGACTCC GCAAAGGACG
CGCAGCCTGC AAGTCCGCGG CCCAGACACA CATTGGGGCT
GACCTGCCGC TGCTGTCAGT GGGAGGCCAG TGGTGCTGGC
CAAGAAGTGT CATGGCTGGT GTCGTGCACG TTTCCCTGGC
TGCTCTCCTC CTGCTGCCTA TGGCCCCTGC CATGCATTCT
GACTGCATCT TCAAGAAGGA GCAAGCCATG TGCCTGGAGA
AGATCCAGAG GGCCAATGAG CTGATGGGCT TCAATGATTC
CTCTCCAGGC TGTCCTGGGA TGTGGGACAA CATCACGTGT
TGGAAGCCCG CCCATGTGGG TGAGATGGTC CTGGTCAGCT
GCCCTGAGCT CTTCCGAATC TTCAACCCAG ACCAAGTCTG
GGAGACCGAA ACCATTGGAG AGTCTGATTT TGGTGACAGT
AACTCCTTAG ATCTCTCAGA CATGGGAGTG GTGAGCCGGA
ACTGCACGGA GGATGGCTGG TCGGAACCCT TCCCTCATTA
CTTTGATGCC TGTGGGTTTG ATGAATATGA ATCTGAGACT
GGGGACCAGG ATTATTACTA CCTGTCAGTG AAGGCCCTCT
ACACGGTTGG CTACAGCACA TCCCTCGTCA CCCTCACCAC
TGCCATGGTC ATCCTTTGTC GCTTCCGGAA GCTGCACTGC
ACACGCAACT TCATCCACAT GAACCTGTTT GTGTCGTTCA
TGCTGAGGGC GATCTCCGTC TTCATCAAAG ACTGGATTCT
GTATGCGGAG CAGGACAGCA ACCACTGCTT CATCTCCACT
GTGGAATGTA AGGCCGTCAT GGTTTTCTTC CACTACTGTG
TTGTGTCCAA CTACTTCTGG CTGTTCATCG AGGGCCTGTA
CCTCTTCACT CTGCTGGTGG AGACCTTCTT CCCTGAAAGG
AGATACTTCT ACTGGTACAC CATCATTGGC TGGGGGACCC
CAACTGTGTG TGTGACAGTG TGGGCTACGC TGAGACTCTA
CTTTGATGAC ACAGGCTGCT GGGATATGAA TGACAGCACA
GCTCTGTGGT GGGTGATCAA AGGCCCTGTG GTTGGCTCTA
TCATGGTTAA CTTTGTGCTT TTTATTGGCA TTATCGTCAT
CCTTGTGCAG AAACTTCAGT CTCCAGACAT GGGAGGCAAT
GAGTCCAGCA TCTACTTGCG ACTGGCCCGG TCCACCCTGC
TGCTCATCCC ACTATTCGGA ATCCACTACA CAGTATTTGC
CTTCTCCCCA GAGAATGTCA GCAAAAGGGA AAGACTCGTG
TTTGAGCTGG GGCTGGGCTC CTTCCAGGGC TTTGTGGTGG
CTGTTCTCTA CTGTTTTCTG AATGGTGAGG TACAAGCGGA
GATCAAGCGA AAATGGCGAA GCTGGAAGGT GAACCGTTAC
TTCGCTGTGG ACTTCAAGCA CCGACACCCG TCTCTGGCCA
GCAGTGGGGT GAATGGGGGC ACCCAGCTCT CCATCCTGAG
CAAGAGCAGC TCCCAAATCC GCATGTCTGG CCTCCCTGCT
GACAATCTGG CCACCTGA

Hop1 ADCYAP1R1 Isoform

(SEQ ID NO:8)

ATGGCTGGTG TCGTGCACGT TTCCCTGGCT GCTCACTGCG
GGGCCTGTCC GTGGGGCCGG GGCAGACTCC GCAAAGGACG
CGCAGCCTGC AAGTCCGCGG CCCAGAGACA CATTGGGGCT
GACCTGCCGC TGCTGTCAGT GGGAGGCCAG TGGTGCTGGC
CAAGAAGTGT CATGCCTGGT GTCGTGCACG TTTCCCTGGC
TGCTCTCCTC CTGCTGCCTA TGGCCCCTGC CATGCATTCT
GACTGCATCT TCAAGAAGGA GCAAGCCATG TGCCTGGAGA
AGATCCAGAG GGCCAATGAG CTGATGGGCT TCAATGATTC
CTCTCCAGGC TGTCCTGCGA TGTGGGACAA CATCACGTGT
TGGAAGCCCG CCCATGTGGG TGAGATGGTC CTGGTCAGCT
GCCCTGAGCT CTTCCGAATC TTCAACCCAG ACCAAGTCTG
GGAGACCGAA ACCATTGGAG AGTCTGATTT TGGTGACAGT
AACTCCTTAG ATCTCTCAGA CATGGGAGTG GTGAGCCCGA
ACTGCACGGA GGATGGCTGG TCGGAACCCT TCCCTCATTA
CTTTGATGCC TGTGGGTTTG ATGAATATGA ATCTGAGACT
GGGGACCAGG ATTATTACTA CCTGTCAGTG AAGGCCCTCT
ACACGGTTGG CTACAGCACA TCCCTCGTCA CCCTCACCAC
TGCCATGGTC ATCCTTTGTC GCTTCCGGAA GCTGCACTGC
ACACGCAACT TCATCCACAT GAACCTGTTT GTGTCGTTCA
TGCTGAGGGC GATCTCCGTC TTCATCAAAG ACTGGATTCT
GTATGCGGAG CAGGACAGGA ACCACTGCTT CATCTCCACT
GTGGAATGTA AGGCCGTCAT GGTTTTCTTC CACTACTGTG
TTGTGTCCAA CTACTTCTGG CTGTTCATCG AGGGCCTGTA
CCTCTTCACT CTGCTGGTGG AGACCTTCTT CCCTGAAAGG
AGATACTTCT ACTGGTACAC CATCATTGGC TGGGGGACCC
CAACTGTGTG TGTGACAGTG TGGGCTACGC TGAGACTCTA
CTTTGATGAC ACAGGCTGCT GGGATATGAA TGACAGCACA
GCTCTGTGGT GGGTGATCAA AGGCCCTGTG GTTGGCTCTA
TCATGGTTAA CTTTGTGCTT TTTATTGGCA TTATCGTCAT
CCTTGTGCAG AAACTTCAGT CTCCAGACAT GGGAGGCAAT
GAGTCCAGCA TCTACTTCAG CTGCGTGCAG AAATGCTACT
GCAAGCCACA GCGGGCTCAG CAGCACTCTT GCAAGATGTC
AGAACTGTCC ACCATTACTC TGCGACTGGC CCGGTCCACC
CTGCTGCTCA TCCCACTATT CGGAATCCAC TACACAGTAT
TTGCCTTCTC CCCAGAGAAT GTCAGCAAAA GGGAAAGACT
CGTGTTTGAG CTGGGGCTGG GCTCCTTCCA GGGCTTTGTG
GTGGCTGTTC TCTACTGTTT TCTGAATGGT GAGGTACAAG
CGGAGATCAA GCGAAAATGG CGAAGCTGGA AGGTGAACCG
TTACTTCGCT GTGGACTTCA AGCACCGACA CCCGTCTCTG
GCCAGCAGTG GGGTGAATGG GGGCACCCAG CTCTCCATCC
TGAGCAAGAG CAGCTCCCAA ATCCGCATGT CTGGCCTCCC
TGCTGACAAT CTGGCCACCT GA

Hop2 ADCYAP1R1 Isoform

(SEQ ID NO:9)

ATGGCTGGTG TCGTGCACGT TTCCCTGGCT GCTCACTGCG
GGGCCTGTCC GTGGGGCCGG GGCAGACTCC GCAAAGGACG
CGCAGCCTGC AAGTCCGCGG CCCAGAGACA CATTGGGGCT
GACCTGCCGC TGCTGTCAGT GGGAGGCCAG TGGTGCTGGC
CAAGAAGTGT CATGGCTGGT GTCGTGCACG TTTCCCTGGC
TGCTCTCCTC CTGCTGCCTA TGGCCCCTGC CATGCATTCT
GACTGCATCT TCAAGAAGGA GCAAGCCATG TGCCTGGAGA
AGATCCAGAG GGCCAATGAG CTGATGGGCT TCAATGATTC
CTCTCCAGCC TGTCCTGGGA TGTGGGACAA CATCACGTGT
TGGAAGCCCG CCCATGTGGG TGAGATGGTC CTGGTCAGCT
GCCCTGAGCT CTTCCGAATC TTCAACCCAG ACCAAGTCTG
GGAGACCGAA ACCATTGGAG AGTCTGATTT TGGTGACAGT
AACTCCTTAG ATCTCTCAGA CATGGGAGTG GTGAGCCGGA
ACTGCACGGA GGATGGCTGG TCGGAACCCT TCCCTCATTA
CTTTGATGCC TGTGGGTTTG ATGAATATGA ATCTGAGACT
GGGGACCAGG ATTATTACTA CCTGTCAGTG AAGGCCCTCT
ACACGGTTGG CTACAGCACA TCCCTCGTCA CCCTCACCAC
TGCCATGGTC ATCCTTTGTC GCTTCCGGAA GCTGCACTGC
ACACGCAACT TCATCCACAT GAACCTGTTT GTGTCGTTCA
TGCTGAGGGC GATCTCCGTC TTCATCAAAG ACTGGATTCT
GTATGCGGAG CAGGACAGCA ACCACTGCTT CATCTCCACT
GTGGAATGTA AGGCCGTCAT GGTTTTCTTC CACTACTGTG
TTGTGTCCAA CTACTTCTGG CTGTTCATCG AGGGCCTGTA
CCTCTTCACT CTGCTGGTGG AGACCTTCTT CCCTGAAAGG
AGATACTTCT ACTGGTACAC CATCATTGGC TGGGGGACCC
CAACTGTGTC TGTGACAGTG TGGGCTACGC TGAGACTCTA
CTTTGATGAC ACAGGCTGCT GGGATATGAA TGACAGCACA
GCTCTGTGGT GGGTGATCAA AGGCCCTGTG GTTGGCTCTA
TCATGGTTAA CTTTGTGCTT TTTATTGCCA TTATCGTCAT
CCTTGTGCAG AAACTTCAGT CTCCAGACAT GGGAGGCAAT
GAGTCCAGCA TCTACTTCTG CGTGCAGAAA TGCTACTGCA
AGCCACAGCG GGCTCAGCAG CACTCTTGCA AGATGTCAGA
ACTGTCCACC ATTACTCTGC GACTGGCCCG GTCCACCCTG
CTGCTCATCC CACTATTCGG AATCCACTAC ACAGTATTTG
CCTTCTCCCC AGAGAATGTC AGCAAAAGGG AAAGACTCGT
GTTTGAGCTG GGGCTGGGCT CCTTCCAGGG CTTTGTGGTG
GCTGTTCTCT ACTGTTTTCT GAATGGTGAG GTACAAGCGG
AGATCAAGCG AAAATGGCGA AGCTGGAAGG TGAACCGTTA
CTTCGCTGTG GACTTCAAGC ACCGACACCC GTCTCTGGCC
AGCAGTGGGG TGAATGGCGG CACCCAGCTC TCCATCCTGA
GCAAGAGCAG CTCCCAAATC CGCATGTCTG GCCTCCCTGC
TGACAATCTG GCCACCTGA

Splice Variants of Adcyaplrl (Mouse)

Short Adcyaplrl Isoform

(SEQ ID NO:10)

ATGGCCAGAA CCCTGCAGCT CTCCCTGACT GCTCTCCTCC
TGCTGCCTAT GGCTATTGCT ATGCACTCTG ACTGCATCTT
CAAGAAGGAG CAAGCCATGT GCCTGGAGAG GATCCAGAGG
GCCAACGACC TGATGGGCCT AAATGAGTCT TCCCCAGGTT
GCCCTGGCAT GTGGGACAAT ATCACATGTT GGAACCCTGC
TCAAATAGGT GAGATGGTCC TTGTGAGCTG CCCTGAGGTC
TTCCGGATCT TCAACCCGGA CCAAGTCTGG ATGACAGAAA
CCATACGGGA TTCTGGCTTT GCTGATAGTA ATTCCTTGGA
GATCACAGAC ATGGGCGTCG TGGGCCGGAA CTGCACTGAG
GATGGCTGGT CGGAGCCCTT CCCCCATTAC TTCGATGCTT
GTGGGTTTGA TGACTATGAG CCCGAGTCTG GGGATCAGGA
TTATTACTAC CTGTCGGTGA AGGCCCTCTA CACAGTCGGC
TACAGCACCT CCCTCGTCAC CCTCACCACT GCCATGGTCA
TCTTGTGCCG CTTCCGGAAG CTGCACTGTA CCCGTAACTT
CATCCACATG AACCTGTTTG TATCCTTCAT GCTGAGAGCT
ATCTCTGTCT TCATCAAAGA CTGGATCTTG TATGCCGAGC
AGGACAGCAG TCATTGCTTC GTTTCCACCG TGGAATGCAA
AGCTGTCATG GTTTTCTTTC ACTACTGCGT GGTGTCCAAC
TACTTCTGGC TGTTCATTGA AGGCCTATAC CTCTTTACAC
TGCTGGTGGA GACCTTCTTC CCTGAGAGGA GATATTTCTA
CTGGTATACC ATCATTGGCT GGGGGACACC TACTGTGTGT
GTAACTGTGT GGGCTGTGCT GAGGCTCTAC TTTGATGATG
CGGGATGCTG GGATATGAAT GACAGCACAG CTCTGTGGTG
GGTGATCAAA GGCCCTGTAG TTGGCTCTAT AATGGTTAAC
TTTGTGCTTT TCATCGGCAT CATCATCATC CTTGTGCAGA
AGCTGCAGTC CCCAGACATG GGAGGCAATG AGTCGAGCAT
CTACTTACGG CTGGCCCGCT CCACCCTGCT GCTCATCCCA
CTCTTTGGAA TCCACTACAC AGTATTTGCC TTCTCTCCAG
AGAACGTCAG CAAGAGGGAA AGACTTGTGT TTGAGCTTGG
GCTGGGCTCC TTCCAGGGCT TTGTGGTGGC TGTACTCTAC
TGCTTCCTGA ATGGGGAGGT ACAGGCAGAG ATTAAGAGGA
AATGGAGGAG CTGGAAGGTG AACCGTTACT TCACTATGGA
CTTCAAGCAC CGGCATCCAT CCCTGGCCAG CAGTGGAGTG
AACGGGGGCA CCCAGCTGTC CATCCTGAGC AAGAGCAGCT
CCCAGCTCCG CATGTCCAGC CTCCCGGCCG ACAACTTGGC
CACCTGA

Hop1 Adcyaplrl Isoform

(SEQ ID NO:11)

ATGCCCAGAA CCCTGCAGCT CTCCCTGACT GCTCTCCTCC
TGCTGCCTAT GGCTATTGCT ATGCACTCTG ACTGCATCTT
CAAGAAGGAG CAAGCCATGT GCCTGGAGAG GATCCAGAGG
GCCAACGACC TGATGGGCCT AAATGAGTCT TCCCCAGGTT
GCCCTGGCAT GTGGGACAAT ATCACATGTT GGAAGCCTGC
TCAAATAGGT GAGATGGTCC TTGTGAGCTG CCCTGAGGTC
TTCCGGATCT TCAACCCGGA CCAAGTCTGG ATGACAGAAA
CCATAGGGGA TTCTGGCTTT GCTGATAGTA ATTCCTTGGA
GATCACAGAC ATGGGGGTCG TGGGCCGGAA CTGCACTGAG
GATGGCTGGT CGGAGCCCTT CCCCCATTAC TTCGATGCTT
GTGGGTTTGA TGACTATGAG CCCGAGTCTG GGGATCAGGA
TTATTACTAC CTGTCGGTGA AGGCCCTCTA CACAGTCGGC
TACAGCACCT CCCTCGTCAC CCTCACCACT GCCATGGTCA
TCTTGTGCCG CTTCCGGAAG CTGCACTGTA CCCGTAACTT
CATCCACATG AACCTGTTTG TATCCTTCAT GCTGAGAGCT
ATCTCTGTCT TCATCAAAGA CTGGATCTTG TATGCCGAGC
AGGACAGCAG TCATTGCTTC GTTTCCACCG TGGAATGCAA
AGCTGTCATG GTTTTCTTTC ACTACTGCGT GGTGTCCAAC
TACTTCTGGC TGTTCATTGA AGGCCTATAC CTCTTTACAC
TGCTGGTGGA GACCTTCTTC CCTGAGAGGA GATATTTCTA
CTGGTATACC ATCATTGGCT GGGGGACACC TACTGTGTGT
GTAACTGTGT GGGCTGTGCT GAGGCTCTAC TTTGATGATG
CGGGATGCTG GGATATGAAT GACACCACAG CTCTGTGGTG
GGTGATCAAA GGCCCTGTAG TTGGCTCTAT AATGGTTAAC
TTTGTGCTTT TCATCGGCAT CATCATCATC CTTGTGCAGA
AGCTGCAGTC CCCAGACATG GGAGGCAATG AGTCGAGCAT
CTACTTCAGC TGCGTGCAGA AATGCTACTG CAAGCCACAG
CGGGCTCAGC AGCACTCTTG CAAGATGTCA GAACTATCCA
CCATTACTCT ACGGCTGGCC CGCTCCACCC TGCTGCTCAT
CCCACTCTTT GGAATCCACT ACACAGTATT TGCCTTCTCT
CCAGAGAACG TCAGCAAGAG GGAAAGACTT GTGTTTGAGC
TTGGGCTGGG CTCCTTCCAG GGCTTTGTGG TGGCTGTACT
CTACTGCTTC CTGAATGGGG AGGTACAGGC AGAGATTAAG
AGGAAATGGA GGAGCTGGAA GGTGAACCGT TACTTCACTA
TGGACTTCAA GCACCGGCAT CCATCCCTGG CCAGCAGTGG
AGTGAACGGG GGCACCCAGC TGTCCATCCT GAGCAAGAGC
AGCTCCCAGC TCCGCATGTC CAGCCTCCCG GCCGACAACT
TGGCCACCTG A

Hop2 Adcyaplrl Isoform

(SEQ ID NO:12)

ATGGCCAGAA CCCTGCAGCT CTCCCTGACT GCTCTCCTCC
TGCTGCCTAT GGCTATTGCT ATGCACTCTG ACTGCATCTT
CAAGAAGGAG CAAGCCATGT GCCTGGAGAG GATCCAGAGG
GCCAACGACC TGATGGGCCT AAATGAGTCT TCCCCAGGTT
GCCCTGGCAT GTGGGACAAT ATCACATGTT GGAAGCCTGC
TCAAATAGGT GAGATGGTCC TTGTGAGCTG CCCTGAGGTC
TTCCGGATCT TCAACCCGGA CCAAGTCTGG ATGACAGAAA
CCATAGGGGA TTCTGGCTTT GCTGATAGTA ATTCCTTGGA
GATCACAGAC ATGGGGGTCG TGGGCCGGAA CTGCACTGAG
GATGGCTGGT CGGAGCCCTT CCCCCATTAC TTCGATGCTT
GTCGGTTTGA TGACTATGAG CCCGAGTCTG GGGATCAGGA
TTATTACTAC CTGTCGGTGA AGGCCCTCTA CACAGTCGGC
TACAGCACCT CCCTCGTCAC CCTCACCACT GCCATGGTCA
TCTTGTGCCG CTTCCGGAAG CTGCACTGTA CCCGTAACTT
CATCCACATG AACCTGTTTG TATCCTTCAT GCTGAGAGCT
ATCTCTGTCT TCATCAAAGA CTCGATCTTG TATGCCGAGC
AGGACAGGAG TCATTGCTTC GTTTCCACCG TGGAATGCAA
AGCTGTCATG GTTTTCTTTC ACTACTGCGT GGTGTCCAAC
TACTTCTGGC TGTTCATTGA AGGCCTATAC CTCTTTACAC
TGCTGGTGGA GACCTTCTTC CCTGAGAGGA GATATTTCTA
CTGGTATACC ATCATTGGCT GGGGGACACC TACTGTGTGT
GTAACTGTGT GGGCTGTGCT GAGGCTCTAC TTTGATGATG
CGGGATGCTG GGATATGAAT GACAGCACAG CTCTGTGGTG
GGTGATCAAA GGCCCTGTAG TTGGCTCTAT AATGGTTAAC
TTTGTGCTTT TCATCGGCAT CATCATCATC CTTGTGCAGA
AGCTGCAGTC CCCAGACATG GGAGGCAATG AGTCGAGCAT
CTACTTCTGC GTGCAGAAAT GCTACTGCAA GCCACAGCGG
GCTCAGCAGC ACTCTTGCAA GATGTCAGAA CTATCCACCA
TTACTCTACG GCTGGCCCGC TCCACCCTGC TGCTCATCCC
ACTCTTTGGA ATCCACTACA CAGTATTTGC CTTCTCTCCA
GAGAACGTCA GCAAGAGGGA AAGACTTGTG TTTGAGCTTG
GGCTGGGCTC CTTCCAGGGC TTTGTGGTGG CTGTACTCTA
CTGCTTCCTG AATGGGGAGG TACAGGCAGA GATTAAGAGG
AAATGGAGGA GCTGGAAGGT GAACCGTTAC TTCACTATGG
ACTTCAAGCA CCGGCATCCA TCCCTGGCCA GCAGTGGAGT
GAACGGGGGC ACCCAGCTGT CCATCCTGAG CAAGAGCAGC
TCCCAGCTCC GCATGTCCAG CCTCCCGGCC GACAACTTGG
CCACCTGA

D. Maxadilan Amino Acid Sequence

CDATCQFRKA IEDCRKKAHH SDVLQTSVQT TATFTSMDTS QLPGSGVFKE CMKEKAKEFK AGK (SEQ ID NO: 13)

References

Altman, J. and G. Das (1965). “Autoradiographic and histological evidence of postnatal hippocampal neurogenesis in rats.” J Comp Neurol 124: 319-335.

Altman, J. and G. Das (1967). “Postnatal neurogenesis in the guinea-pig.” Nature 214: 1098-1101.

Arimura, A. (1998). “Perspectives on pituitary adenylate cyclase activating polypeptide (PACAP) in the neuroendocrine, endocrine, and nervous systems.” Jpn J Physiol 48(5): 301-31.

Biebl, M., C. M. Cooper, et al. (2000). “Analysis of neurogenesis and programmed cell death reveals a self-renewing capacity in the adult rat brain.” Neurosci Lett 291(1): 17-20.

Bjorklund, A. and O. Lindvall (2000). “Cell replacement therapies for central nervous system disorders.” Nat Neurosci 3(6): 537-44.

Craig, C. G., V. Tropepe, et al. (1996). “In vivo growth factor expansion of endogenous subependyimal neural precursor cell populations in the adult mouse brain.” J Neurosci 16(8): 2649-58.

Crouch, S. P., R. Kozlowski, et al. (1993). “The use of ATP bioluminescence as a measure of cell proliferation and cytotoxicity.” J Immunol Methods 160(1): 81-8.

Doetsch, F., I. Caille, et al. (1999). “Subventricular zone astrocytes are neural stem cells in the adult mammalian brain.” Cell 97(6): 703-16.

Feany, M. B. and W. G. Quinn (1995). “A neuropeptide gene defined by the Drosophila memory mutant amnesiac.” Science 268(5212): 869-73.

Gage, F. H., G. Kempermann, et al. (1998). “Multipotent progenitor cells in the adult dentate gyrus.” J Neurobiol 36(2): 249-66.

Ghatei, M. A., K. Takahashi, et al. (1993). “Distribution, molecular characterization of pituitary adenylate cyclase-activating polypeptide and its precursor encoding messenger RNA in human and rat tissues.” J Endocrinol 136(1): 159-66.

Gottschall, P. E., I. Tatsuno, et al. (1994). “Regulation of interleukin-6 (IL-6) secretion in primary cultured rat astrocytes: synergism of interleukin-1 (IL-1) and pituitary adenylate cyclase activating polypeptide (PACAP).” Brain Res 637(1-2): 197-203.

Gressens, P. (1999). “VIP neuroprotection against excitotoxic lesions of the developing mouse brain.” Ann NY Acad Sci 897: 109-24.

Gressens, P., B. Paindaveine, et al. (1997). “Growth factor properties of VIP during early brain development. Whole embryo culture and in vivo studies.” Ann NY Acad Sci 814: 152-60.

Hansel, D. E., B. A. Eipper, et al. (2001). “Regulation of olfactory neurogenesis by amidated neuropeptides.” J Neurosci Res 66(1): 1-7.

Hansel, D. E., V. May, et al. (2001). “Pituitary adenylyl cyclase-activating peptides and alpha-amidation in olfactory neurogenesis and neuronal survival in vitro.” J Neurosci 21(13): 4625-36.

Hashimoto, H., T. Ishihara, et al. (1993). “Molecular cloning and tissue distribution of a receptor for pituitary adenylate cyclase-activating polypeptide.” Neuron 11(2): 333-42.

Hashimoto, H., H. Nogi, et al. (1996). “Distribution of the mRNA for a pituitary adenylate cyclase-activating polypeptide receptor in the rat brain: an in situ hybridization study.” J Comp Neurol 371(4): 567-77.

Herman, J. P. and N. D. Abrous (1994). “Dopaminergic neural grafts after fifteen years: results and perspectives.” Prog Neurobiol 44(1): 1-35.

Jacobson, M. (1991). Histosenesis and morphogenesis of cortical structures. Developmental Neurobiology, Plenum Press, New York: 401-451.

Jaworski, D. M. and M. D. Proctor (2000). “Developmental regulation of pituitary adenylate cyclase-activating polypeptide and PAC(1) receptor mRNA expression in the rat central nervous system.” Brain Res Dev Brain Res 120(1): 27-39.

Johansson, C. B., S. Momma, et al. (1 999). “Identification of a neural stem cell in the adult mammalian central nervous system.” Cell 96(1): 25-34.

Johansson, C. B., M. Svensson, et al. (1999). “Neural stem cells in the adult human brain.” Exp Cell Res 253(2): 733-6.

Johe, K. K., T. G. Hazel, et al. (1996). “Single factors direct the differentiation of stem cells from the fetal and adult central nervous system.” Genes Dev 10(24): 3129-40.

Kuhn, H. G. and C. N. Svendsen (1999). “Origins, functions, and potential of adult neural stem cells.” Bioessays 21(8): 625-30.

Kuhn, H. G., J. Winkler, et al. (1997). “Epidermal growth factor and fibroblast growth factor-2 have different effects on neural progenitors in the adult rat brain.” J Neurosci 17(15): 5820-9.

Lerner, E. A., J. M. Ribeiro, et al. (1991). “Isolation of maxadilan, a potent vasodilatory peptide from the salivary glands of the sand fly Lutzomyia longipalpis.” J Biol Chem 266(17): 11234-6.

Lois, C. and A. Alvarez-Buylla (1993). “Proliferating subventricular zone cells in the adult mammalian forebrain can differentiate into neurons and glia.” Proc Natl Acad Sci USA 90(5): 2074-7.

Magavi, S. S., B. R. Leavitt, et al. (2000). “Induction of neurogenesis in the neocortex of adult mice [see comments].” Nature 405(6789): 951-5.

Masuo, Y., N. Suzuki, et al. (1993). “Regional distribution of pituitary adenylate cyclase activating polypeptide (PACAP) in the rat central nervous system as determined by sandwich-enzyme immunoassay.” Brain Res 602(1): 57-63.

McKay, R. (1997). “Stem cells in the central nervous system.” Science 276(5309): 66-71.

Momma, S., C. B. Johansson, et al. (2000). “Get to know your stem cells.” Curr Opin Neurobiol 10(1): 45-9.

Moore, M. S., J. DeZazzo, et al. (1998). “Ethanol intoxication in Drosophila: Genetic and pharmacological evidence for regulation by the cAMP signaling pathway.” Cell 93(6): 997-1007.

Morio, H., I. Tatsuno, et al. (1996). “Pituitary adenylate cyclase-activating polypeptide protects rat-cultured cortical neurons from glutamate-induced cytotoxicity.” Brain Res 741(1-2): 82-8.

Moro, O. and E. A. Lerner (1997). “Maxadilan, the vasodilator from sand flies, is a specific pituitary adenylate cyclase activating peptide type I receptor agonist.” J Biol Chem 272(2): 966-70.

Moser, A., J. Scholz, et al. (1999). “Pituitary adenylate cyclase-activating polypeptide (PACAP-27) enhances tyrosine hydroxylase activity in the nucleus accumbens of the rat.” Neuropeptides 33(6): 492-7.

Nakagawa, S., J.-E. Kim, et al. (2002). “Regulation of neurogenesis in adult mouse hippocampus by cAMP and the cAMP response element-binding protein.” J Neurosci 22(9): 3673-3682.

Palmer, T. D., E. A. Markakis, et al. (1999). “Fibroblast growth factor-2 activates a latent neurogenic program in neural stem cells from diverse regions of the adult CNS.” J Neurosci 19(19): 8487-97.

Patrone, C., S. Andersson, et al. (1999). “Estrogen receptor-dependent regulation of sensory neuron survival in developing dorsal root ganglion.” Proc Natl Acad Sci USA 96(19): 10905-10.

Pencea, V., K. D. Bingaman, et al. (2001). “Infusion of Brain-Derived Neurotrophic Factor into the Lateral Ventricle of the Adult Rat Leads to New Neurons in the Parenchyma of the Striatum, Septum, Thalamus, and Hypothalamus.” J Neurosci 21(17): 6706-17.

Piggins, H. D., J. A. Stamp, et al. (1996). “Distribution of pituitary adenylate cyclase activating polypeptide (PACAP) immunoreactivity in the hypothalamus and extended amygdala of the rat.” J Comp Neurol 376(2): 278-94.

Rajan, P. and R. D. McKay (1998). “Multiple routes to astrocytic differentiation in the CNS.” J Neurosci 18(10): 3620-9.

Snyder, E. Y., C. Yoon, et al. (1997). “Multipotent neural precursors can differentiate toward replacement of neurons undergoing targeted apoptotic degeneration in adult mouse neocortex.” Proc Natl Acad Sci USA 94(21): 11663-8.

Takel, N., Y. Skoglosa, et al. (1998). “Neurotrophic and neuroprotective effects of pituitary adenylate cyclase-activating polypeptide (PACAP) on mesencephalic dopaminergic neurons.” J Neurosci Res 54(5): 698-706.

Tyrrell, S. and S. C. Landis (1994). “The appearance of NPY and VIP in sympathetic neuroblasts and subsequent alterations in their expression.” J Neurosci 14(7): 4529-47.

Usdin, T. B., T. I. Bonner, et al. (1994). “Two receptors for vasoactive intestinal polypeptide with similar specificity and complementary distributions.” Endocrinology 135(6): 2662-80.

Vaudry, D., B. J. Gonzalez, et al. (2000). “Pituitary adenylate cyclase-activating polypeptide and its receptors: from structure to functions.” Pharmacol Rev 52(2): 269-324.

Vaudry, D., P. J. Stork, et al. (2002). “Signaling pathways for PC12 cell differentiation: making the right connections.” Science 296(5573): 1648-9.

Villalba, M., J. Bockaert, et al. (1997). “Pituitary adenylate cyclase-activating polypeptide (PACAP-38) protects cerebellar granule neurons from apoptosis by activating the mitogen-activated protein kinase (MAP kinase) pathway.” J Neurosci 17(1): 83-90.

Williams, B. P., J. K. Park, et al. (1997). “A PDGF-regulated immediate early gene response initiates neuronal differentiation in ventricular zone progenitor cells.” Neuron 18(4): 553-62.

Zhang, R. L., Z. G. Zhang, et al. (2001). “Proliferation and differentiation of progenitor cells in the cortex and the subventricular zone in the adult rat after focal cerebral ischemia.” Neuroscience 105(1): 33-41.

Zhong, Y. (1995). “Mediation of PACAP-like neuropeptide transmission by coactivation of Ras/Raf and cAMP signal transduction pathways in Drosophila.” Nature 375(6532): 588-92.

We claim:

1. A method of alleviating a symptom of a disorder of the nervous system in a patient comprising administering PACAP, Maxadilan, PACAP receptor agonist, ADCYAP1R1 agonist or a combination thereof to modulate NSC activity in vivo to a patient suffering from the disease or disorder of the nervous system.

2. The method of claim 1 wherein the NSC activity is proliferation, differentiation, migration or survival.

3. The method of claim 1 wherein the PACAP, Maxadilan, PACAP receptor agonist, ADCYAP1R1 agonist or a combination therof is administered in an amount of 0.001 ng/kg/day to 10 mg/kg/day.

4. The method of claim 1 wherein the PACAP, Maxadilan, PACAP receptor agonist, ADCYAP1R1 agonist is administered to achieve a target tissue concentration of 0.01 nM to 1 μM.

5. The method of claim 4 wherein the target tissue is selected from the group consisting of the ventricular wall, the volume adjacent to the wall of the ventricular system, hippocampus, alveus, striatum, substantia nigra, retina, nucleus basalis of Meynert, spinal cord, thalamus, hypothalamus and cortex.

6. The method of claim 1 wherein the PACAP, Maxadilan, PACAP receptor agonist, ADCYAP1R1 agonist is administered by injection.

7. The method of claim 6 wherein the injection is given subcutaneously, intraperitoneally, intramusclularly, intracerebroventricularly, intraparenchymally, intrathecally or intracranially.

8. The method of claim 1 wherein the PACAP, Maxadilan, PACAP receptor agonist, ADCYAP1R1 agonist is administered orally.

9. The method of claim 1 wherein the disease or disorder of the nervous system is selected from the group consisting of neurodegenerative disorders, NSC disorders, neural progenitor disorders, ischemic disorders, neurological traumas, affective disorders, neuropsychiatric disorders, degenerative diseases of the retina, retinal injury/trauma, cognitive performance and learning and memory disorders.

10. A method of modulating the activity of a receptor for PACAP, or Maxadilan or a combination thereof, on a NSC comprising the step of exposing the cell expressing the receptor to a modulator agent, wherein the exposure induces an NSC to proliferate, differentiate, migrate or survive.

11. The method of claim 10 wherein the modulator agent is an exogenous reagent, an antibody, an affibody or a combination thereof.

12. The method of claim 10 wherein the PACAP receptor is ADCYAP1R1, VIPR1 or VIPR2.

13. The method of claim 10 wherein the Maxadilan receptor is ADCYAP1R1.

14. The method of claim 11 wherein the modulator agent is selected from the group consisting of PACAP, Maxadilan, PACAP receptor agonist, and ADCYAP1R1 agonist.

15. The method of claim 11 wherein the modulator agent is pegylated.

16. The method of claim 11 wherein the antibody is a monoclonal or a polyclonal antibody.

17. The method of claim 10 wherein the NSC is derived from fetal brain, adult brain, neural cell culture or a neurosphere.

18. The method of claim 10 wherein the NSC is derived from tissue enclosed by dura mater, peripheral nerves or ganglia.

19. The method of claim 10 wherein the NSC is derived from stem cells originating from a tissue selected from the group consisting of pancreas, skin, muscle, adult bone marrow, liver, umbilical cord tissue and umbilical cord blood.

20. A method for stimulating mammalian adult NSC proliferation or neurogenesis comprising the step of contacting a cell population comprising mammalian adult NSC to a agent selected from the group consisting of PACAP, Maxadilan, PACAP receptor agonist, and ADCYAP1R1 agonist to form a treated NSC, wherein the treated NSC cell shows improved proliferation or neurogenesis compared to untreated cells.

21. The method of claim 20 wherein the NSC is derived from lateral ventricle wall of a mammalian brain.

22. The method of claim 20 wherein the NSC is derived from stem cells originating from a tissue selected from the group consisting of pancreas, skin, muscle, adult bone marrow, liver, umbilical cord tissue and umbilical cord blood.

23. The method of claim 20 wherein the treated NSC shows improved differentiation, survival or migration compared to untreated cells.

24. A method for synergistically stimulating mammalian adult NSC proliferation or neurogenesis comprising the step of contacting a cell population comprising mammalian adult neural stem cells to a growth factor and an agent selected from the group consisting of PACAP, Maxadilan, PACAP receptor agonist, and ADCYAP1R1 agonist.

25. The method of claim 24, wherein the stimulation of mammalian adult NSC proliferation is greater than stimulation by the growth factor or stimulation by the agent alone.

26. The method of claim 24, wherein the stimulation of mammalian adult NSC proliferation is greater than the sum of stimulation by growth factor and stimulation by the agent.

27. The method of claim 24 wherein the growth factor is EGF.

28. A method for stimulating mammalian adult NSC proliferation comprising the step of contacting a cell population comprising mammalian adult NSC to VEGF and an agent selected from the group consisting of PACAP, Maxadilan, PACAP receptor agonist, and ADCYAP1R1 agonist.

29. A method for inducing NSC proliferation comprising the step of increasing intracellular CREB phosphorylation.

30. The method of claim 29 wherein the step of increasing intracellualar CREB phosphorylation involves contacting the NSC with an agent selected from the group consisting of PACAP, Maxadilan, PACAP receptor agonist, and ADCYAP1R1 agonist.

31. A method for inducing NSC proliferation comprising the step of increasing intracellular AP-1 transcription.

32. The method of claim 31 wherein the step of increasing intracellular AP-1 transcription involves contacting the NSC with an agent selected from the group consisting of PACAP, Maxadilan, PACAP receptor agonist, and ADCYAP1R1 agonist.

33. A method for inducing NSC proliferation comprising the step of increasing intracellular protein kinase C activity.

34. The method of claim 33 wherein the step of increasing intracellular protein kinase C activity involves contacting the NSC with an agent selected from the group consisting of PACAP, Maxadilan, PACAP receptor agonist, and ADCYAP1R1 agonist.

35. A method for stimulating survival of mammalian adult NSC progeny comprising the step of increasing intracellular CREB phosphorylation in the mammalian adult NSC progeny.

36. The method of claim 35 wherein the step of increasing intracellular CREB phosphorylation comprises contacting the NSC with an agent selected from the group consisting of PACAP, Maxadilan, PACAP receptor agonist, and ADCYAP1R1 agonist.

37. A method for stimulating primary adult mammalian NSC to proliferate to form neurospheres comprising contacting the cell with an agent selected from the group consisting of PACAP, Maxadilan, PACAP receptor agonist, and ADCYAP1R1 agonist to produce a proliferating NSC.

38. A method for inducing the in situ proliferation, differentiation, migration or survival of an NSC located in the neural tissue of a mammal, the method comprising administering a therapeutically effective amount of PACAP, Maxadilan, PACAP receptor agonist, or ADCYAP1R1 agonist to the neural tissue to induce the proliferation, differentiation, migration or survival of the cell.

39. A method for accelerating the growth of an NSC in a desired target tissue in a subject, comprising:
(a) transfecting the target tissue with an expression vector containing an open reading frame encoding PACAP, Maxadilan, VEPR1, VIPR2 or ADCYAP1R1 gene in a therapeutically effective amount;
(b) expressing the open reading frame to produce a protein in the target tissue.

40. The method of claim 39 wherein the transfecting step involves administration of the expression vector by injection.

41. The method of claim 39 wherein the expression vector is a non-viral expression vector encapsulated in a liposome.

42. A method of enhancing neurogenesis in a patient suffering from a central nervous system disorder comprising the step of infusing PACAP, Maxadilan, PACAP receptor agonist, or ADCYAP1R1 agonist thereof into the patient.

43. The method of claim 42 wherein the infusion is selected from the group consisting of intraventricular, intravenous, sublingual, subcutaneous and intraarterial infusion.

44. A method of alleviating a symptom of a central nervous system disorder in a patient comprising the step of infusing PACAP, Maxadilan, PACAP receptor agonist, and ADCYAP1R1 agonist into the patient.

45. A method for producing a cell population enriched for human NSC, comprising:
(a) contacting a cell population containing NSC with a reagent that recognizes a determinant on a PACAP receptor, a Maxadilan receptor, or an ADCYAP1R1;
(b) selecting for cells in which there is contact between the reagent and the determinant on the surface of the cells of step (a) to produce a population highly enriched for central nervous system stem cells.

46. The method of claim 45 wherein the reagent is selected from the group consisting of a small molecule, a peptide, an antibody and an affibody.

47. The method of claim 45 wherein the population containing NSC are obtained from neural tissue.

48. The method of claim 45 wherein the cell population is derived from whole mammalian fetal brain or whole mammalian adult brain.

49. The method of claim 45 wherein the human NSCs are derived from stem cells originating from a tissue selected from the group consisting of pancreas, skin, muscle, adult bone marrow, liver, umbilical cord tissue and umbilical cord blood.

50. An in vitro cell culture comprising a cell population generated by the method of claim 45 wherein the cell population is enriched for cells expressing receptors selected from the group consisting of ADCYAP1R1, VIPR1 or VIPR2.

51. A method for alleviating a symptom of a central nervous system disorder comprising administering the population of claim 50 to a mammal in need thereof.

52. A method of reducing a symptom of a central nervous system disorder in a patient comprising the step of administering into the spinal cord of the subject a composition comprising
(a) a population of isolated NSCs obtained from fetal or adult tissue; and
(b) PACAP, Maxadilan, PACAP receptor agonist, ADCYAP1R1 agonist or a combination thereof;
whereby the symptom is reduced.

53. A method of reducing a symptom of a central nervous disorder in a patient comprising the steps of
(a) introducing a viral vector into the target cell, wherein the viral vector has at least one insertion site containing a nucleic acid which encodes PACAP, Maxadilan, PACAP receptor agonist, or ADCYAP1R1 agonist, the nucleic acid gene operably linked to a promoter capable of expression in the host
(b) expressing the nucleic acid to produce a protein in a target cell to reduce the symptom.

54. A method for alleviating a symptom of a disease or disorder of the nervous system in a patient comprising the steps of:
(a) providing a population of NSC;
(b) suspending the NSC in a solution comprising PACAP, Maxadilan, PACAP receptor agonist, ADCYAP1R1 agonist or a combination thereof to generate a cell suspension;
(c) delivering the cell suspension to an injection site in the nervous system of the patient to alleviate the symptom.

55. The method of claim 54 further comprising the step of administering to the injection site a growth factor for a period of time before the step of delivering the cell suspension.

56. The method of claim 54 further comprising the step of administering to the injection site a growth factor after the delivering step.

57. A method for transplanting a population of cells enriched for human NSC, comprising:
(a) contacting a population containing NSC with a reagent that recognizes a determinant on a PACAP receptor, a Maxadilan receptor, or an ADCYAP1R1;
(b) selecting for cells in which there is contact between the reagent and the determinant on the surface of the cells of step (a), to produce a population highly enriched for central nervous system stem cells; and
(c) implanting the selected cells of step (b) into a non-human mammal.

58. A method of modulating a Maxadilan receptor agonist, PACAP receptor agonist, or ADCYAP1R1 agonist on the surface of an NSC comprising the step of contacting the cell expressing the receptor to exogenous reagent, antibody, or affibody, wherein the exposure induces the NSC to proliferate, differentiate, migrate or survive.

59. The method of claim 58 wherein the NSC is derived from fetal brain, adult brain, neural cell culture or a neurosphere.

60. A method of determining an isolated candidate PACAP or Maxadilan receptor modulator compound for its ability to modulate NSC activity comprising the steps of:
(a) administering the isolated candidate compound to a non-human mammal; and
(b) determining if the candidate compound has an effect on modulating the NSC activity in the non-human mammal.

61. The method of claim 60 wherein the determining step comprises comparing the neurological effects of the non-human mammal with a referenced non-human mammal not administered the candidate compound.

62. The method of claim 60 wherein the NSC activity is proliferation, differentiation, migration or survival.

63. The method of claim 60 wherein the PACAP or Maxadilan receptor modulator is administered by injection.

64. The method of claim 63 wherein the injection is given subcutaneously, intraperitoneally, intramuscluarly, intracerebroventricularly, intraparenchymally, intrathecally or intracranially.

65. The method of claim 60 wherein the PACAP or Maxadilan receptor modulator is administered via peptide fusion or micelle delivery.


SEQUENCE LISTING (13 sequences)

SEQ ID NO: 1 - DNA, 25 bp, Mus musculus
cctgtcggtgaaggccctctacaca

SEQ ID NO: 2 - DNA, 25 bp, Mus musculus
cccagcccaagctcaaacacaagtc

SEQ ID NO: 3 - DNA, 22 bp, Mus musculus
tactttgatgatgcgggatgct

SEQ ID NO: 4 - DNA, 22 bp, Mus musculus
agtacagccaccacaaagccct

SEQ ID NO: 5 - DNA, 22 bp, Mus musculus
tactttgatgacacaggctgct

SEQ ID NO: 6 - DNA, 22 bp, Mus musculus
agtacagccaccacaaagccct

SEQ ID NO: 7 - DNA, 1578 bp, Homo sapiens
atggctggtgtcgtgcacgtttccctggctgctcactgcggggcctgtccgtggggccggggcagactccgcaaaggacgcgcagcctgcaagtccgcggcccagagacacattggggctgacctgccgctgctgtcagtgggaggccagtggtgctggccaagaagtgtcatggctggtgtcgtgcacgtttccctggctgctctcctcctgctgcctatggcccctgccatgcattctgactgcatcttcaagaaggagcaagccatgtgcctggagaagatccagagggccaatgagctgatgggcttcaatgattcctctccaggctgtcctgggatgtgggacaacatcacgtgttggaagcccgcccatgtgggtgagatggtcctggtcagctgccctgagctcttccgaatcttcaacccagaccaagtctgggagaccgaaaccattggagagtctgattttggtgacagtaactccttagatctctcagacatgggagtggtgagccggaactgcacggaggatggctggtcggaacccttccctcattactttgatgcctgtgggtttgatgaatatgaatctgagactggggaccaggattattactacctgtcagtgaaggccctctacacggttggctacagcacatccctcgtcaccctcaccactgccatggtcatcctttgtcgcttccggaagctgcactgcacacgcaacttcatccacatgaacctgtttgtgtcgttcatgctgagggcgatctccgtcttcatcaaagactggattctgtatgcggagcaggacagcaaccactgcttcatctccactgtggaatgtaaggccgtcatggttttcttccactactgtgttgtgtccaactacttctggctgttcatcgagggcctgtacctcttcactctgctggtggagaccttcttccctgaaaggagatacttctactggtacaccatcattggctgggggaccccaactgtgtgtgtgacagtgtgggctacgctgagactctactttgatgacacaggctgctgggatatgaatgacagcacagctctgtggtgggtgatcaaaggccctgtggttggctctatcatggttaactttgtgctttttattggcattatcgtcatccttgtgcagaaacttcagtctccagacatgggaggcaatgagtccagcatctacttgcgactggcccggtccaccctgctgctcatcccactattcggaatccactacacagtatttgccttctccccagagaatgtcagcaaaagggaaagactcgtgtttgagctggggctgggctccttccagggctttgtggtggctgttctctactgttttctgaatggtgaggtacaagcggagatcaagcgaaaatggcgaagctggaaggtgaaccgttacttcgctgtggacttcaagcaccgacacccgtctctggccagcagtggggtgaatgggggcacccagctctccatcctgagcaagagcagctcccaaatccgcatgtctggcctccctgctgacaatctggccacctga

SEQ ID NO: 8 - DNA, 1662 bp, Homo sapiens
atggctggtgtcgtgcacgtttccctggctgctcactgcggggcctgtccgtggggccggggcagactccgcaaaggacgcgcagcctgcaagtccgcggcccagagacacattggggctgacctgccgctgctgtcagtgggaggccagtggtgctggccaagaagtgtcatggctggtgtcgtgcacgtttccctggctgctctcctcctgctgcctatggcccctgccatgcattctgactgcatcttcaagaaggagcaagccatgtgcctggagaagatccagagggccaatgagctgatgggcttcaatgattcctctccaggctgtcctgggatgtgggacaacatcacgtgttggaagcccgcccatgtgggtgagatggtcctggtcagctgccctgagctcttccgaatcttcaacccagaccaagtctgggagaccgaaaccattggagagtctgattttggtgacagtaactccttagatctctcagacatgggagtggtgagccggaactgcacggaggatggctggtcggaacccttccctcattactttgatgcctgtgggtttgatgaatatgaatctgagactggggaccaggattattactacctgtcagtgaaggccctctacacggttggctacagcacatccctcgtcaccctcaccactgccatggtcatcctttgtcgcttccggaagctgcactgcacacgcaacttcatccacatgaacctgtttgtgtcgttcatgctgagggcgatctccgtcttcatcaaagactggattctgtatgcggagcaggacagcaaccactgcttcatctccactgtggaatgtaaggccgtcatggttttcttccactactgtgttgtgtccaactacttctggctgttcatcgagggcctgtacctcttcactctgctggtggagaccttcttccctgaaaggagatacttctactggtacaccatcattggctgggggaccccaactgtgtgtgtgacagtgtgggctacgctgagactctactttgatgacacaggctgctgggatatgaatgacagcacagctctgtggtgggtgatcaaaggccctgtggttggctctatcatggttaactttgtgctttttattggcattatcgtcatccttgtgcagaaacttcagtctccagacatgggaggcaatgagtccagcatctacttcagctgcgtgcagaaatgctactgcaagccacagcgggctcagcagcactcttgcaagatgtcagaactgtccaccattactctgcgactggcccggtccaccctgctgctcatcccactattcggaatccactacacagtatttgccttctccccagagaatgtcagcaaaagggaaagactcgtgtttgagctggggctgggctccttccagggctttgtggtggctgttctctactgttttctgaatggtgaggtacaagcggagatcaagcgaaaatggcgaagctggaaggtgaaccgttacttcgctgtggacttcaagcaccgacacccgtctctggccagcagtggggtgaatgggggcacccagctctccatcctgagcaagagcagctcccaaatccgcatgtctggcctccctgctgacaatctggccacctga

SEQ ID NO: 9 - DNA, 1659 bp, Homo sapiens
atggctggtgtcgtgcacgtttccctggctgctcactgcggggcctgtccgtggggccggggcagactccgcaaaggacgcgcagcctgcaagtccgcggcccagagacacattggggctgacctgccgctgctgtcagtgggaggccagtggtgctggccaagaagtgtcatggctggtgtcgtgcacgtttccctggctgctctcctcctgctgcctatggcccctgccatgcattctgactgcatcttcaagaaggagcaagccatgtgcctggagaagatccagagggccaatgagctgatgggcttcaatgattcctctccaggctgtcctgggatgtgggacaacatcacgtgttggaagcccgcccatgtgggtgagatggtcctggtcagctgccctgagctcttccgaatcttcaacccagaccaagtctgggagaccgaaaccattggagagtctgattttggtgacagtaactccttagatctctcagacatgggagtggtgagccggaactgcacggaggatggctggtcggaacccttccctcattactttgatgcctgtgggtttgatgaatatgaatctgagactggggaccaggattattactacctgtcagtgaaggccctctacacggttggctacagcacatccctcgtcaccctcaccactgccatggtcatcctttgtcgcttccggaagctgcactgcacacgcaacttcatccacatgaacctgtttgtgtcgttcatgctgagggcgatctccgtcttcatcaaagactggattctgtatgcggagcaggacagcaaccactgcttcatctccactgtggaatgtaaggccgtcatggttttcttccactactgtgttgtgtccaactacttctggctgttcatcgagggcctgtacctcttcactctgctggtggagaccttcttccctgaaaggagatacttctactggtacaccatcattggctgggggaccccaactgtgtgtgtgacagtgtgggctacgctgagactctactttgatgacacaggctgctgggatatgaatgacagcacagctctgtggtgggtgatcaaaggccctgtggttggctctatcatggttaactttgtgctttttattggcattatcgtcatccttgtgcagaaacttcagtctccagacatgggaggcaatgagtccagcatctacttctgcgtgcagaaatgctactgcaagccacagcgggctcagcagcactcttgcaagatgtcagaactgtccaccattactctgcgactggcccggtccaccctgctgctcatcccactattcggaatccactacacagtatttgccttctccccagagaatgtcagcaaaagggaaagactcgtgtttgagctggggctgggctccttccagggctttgtggtggctgttctctactgttttctgaatggtgaggtacaagcggagatcaagcgaaaatggcgaagctggaaggtgaaccgttacttcgctgtggacttcaagcaccgacacccgtctctggccagcagtggggtgaatgggggcacccagctctccatcctgagcaagagcagctcccaaatccgcatgtctggcctccctgctgacaatctggccacctga

SEQ ID NO: 10 - DNA, 1407 bp, Mus musculus
atggccagaaccctgcagctctccctgactgctctcctcctgctgcctatggctattgctatgcactctgactgcatcttcaagaaggagcaagccatgtgcctggagaggatccagagggccaacgacctgatgggcctaaatgagtcttccccaggttgccctggcatgtgggacaatatcacatgttggaagcctgctcaaataggtgagatggtccttgtgagctgccctgaggtcttccggatcttcaacccggaccaagtctggatgacagaaaccataggggattctggctttgctgatagtaattccttggagatcacagacatgggggtcgtgggccggaactgcactgaggatggctggtcggagcccttcccccattacttcgatgcttgtgggtttgatgactatgagcccgagtctggggatcaggattattactacctgtcggtgaaggccctctacacagtcggctacagcacctccctcgtcaccctcaccactgccatggtcatcttgtgccgcttccggaagctgcactgtacccgtaacttcatccacatgaacctgtttgtatccttcatgctgagagctatctctgtcttcatcaaagactggatcttgtatgccgagcaggacagcagtcattgcttcgtttccaccgtggaatgcaaagctgtcatggttttctttcactactgcgtggtgtccaactacttctggctgttcattgaaggcctatacctctttacactgctggtggagaccttcttccctgagaggagatatttctactggtataccatcattggctgggggacacctactgtgtgtgtaactgtgtgggctgtgctgaggctctactttgatgatgcgggatgctgggatatgaatgacagcacagctctgtggtgggtgatcaaaggccctgtagttggctctataatggttaactttgtgcttttcatcggcatcatcatcatccttgtgcagaagctgcagtccccagacatgggaggcaatgagtcgagcatctacttacggctggcccgctccaccctgctgctcatcccactctttggaatccactacacagtatttgccttctctccagagaacgtcagcaagagggaaagacttgtgtttgagcttgggctgggctccttccagggctttgtggtggctgtactctactgcttcctgaatggggaggtacaggcagagattaagaggaaatggaggagctggaaggtgaaccgttacttcactatggacttcaagcaccggcatccatccctggccagcagtggagtgaacgggggcacccagctgtccatcctgagcaagagcagctcccagctccgcatgtccagcctcccggccgacaacttggccacctga

SEQ ID NO: 11 - DNA, 1491 bp, Mus musculus
atggccagaaccctgcagctctccctgactgctctcctcctgctgcctatggctattgctatgcactctgactgcatcttcaagaaggagcaagccatgtgcctggagaggatccagagggccaacgacctgatgggcctaaatgagtcttccccaggttgccctggcatgtgggacaatatcacatgttggaagcctgctcaaataggtgagatggtccttgtgagctgccctgaggtcttccggatcttcaacccggaccaagtctggatgacagaaaccataggggattctggctttgctgatagtaattccttggagatcacagacatgggggtcgtgggccggaactgcactgaggatggctggtcggagcccttcccccattacttcgatgcttgtgggtttgatgactatgagcccgagtctggggatcaggattattactacctgtcggtgaaggccctctacacagtcggctacagcacctccctcgtcaccctcaccactgccatggtcatcttgtgccgcttccggaagctgcactgtacccgtaacttcatccacatgaacctgtttgtatccttcatgctgagagctatctctgtcttcatcaaagactggatcttgtatgccgagcaggacagcagtcattgcttcgtttccaccgtggaatgcaaagctgtcatggttttctttcactactgcgtggtgtccaactacttctggctgttcattgaaggcctatacctctttacactgctggtggagaccttcttccctgagaggagatatttctactggtataccatcattggctgggggacacctactgtgtgtgtaactgtgtgggctgtgctgaggctctactttgatgatgcgggatgctgggatatgaatgacagcacagctctgtggtgggtgatcaaaggccctgtagttggctctataatggttaactttgtgcttttcatcggcatcatcatcatccttgtgcagaagctgcagtccccagacatgggaggcaatgagtcgagcatctacttcagctgcgtgcagaaatgctactgcaagccacagcgggctcagcagcactcttgcaagatgtcagaactatccaccattactctacggctggcccgctccaccctgctgctcatcccactctttggaatccactacacagtatttgccttctctccagagaacgtcagcaagagggaaagacttgtgtttgagcttgggctgggctccttccagggctttgtggtggctgtactctactgcttcctgaatggggaggtacaggcagagattaagaggaaatggaggagctggaaggtgaaccgttacttcactatggacttcaagcaccggcatccatccctggccagcagtggagtgaacgggggcacccagctgtccatcctgagcaagagcagctcccagctccgcatgtccagcctcccggccgacaacttggccacctga

SEQ ID NO: 12 - DNA, 1488 bp, Mus musculus
atggccagaaccctgcagctctccctgactgctctcctcctgctgcctatggctattgctatgcactctgactgcatcttcaagaaggagcaagccatgtgcctggagaggatccagagggccaacgacctgatgggcctaaatgagtcttccccaggttgccctggcatgtgggacaatatcacatgttggaagcctgctcaaataggtgagatggtccttgtgagctgccctgaggtcttccggatcttcaacccggaccaagtctggatgacagaaaccataggggattctggctttgctgatagtaattccttggagatcacagacatgggggtcgtgggccggaactgcactgaggatggctggtcggagcccttcccccattacttcgatgcttgtgggtttgatgactatgagcccgagtctggggatcaggattattactacctgtcggtgaaggccctctacacagtcggctacagcacctccctcgtcaccctcaccactgccatggtcatcttgtgccgcttccggaagctgcactgtacccgtaacttcatccacatgaacctgtttgtatccttcatgctgagagctatctctgtcttcatcaaagactggatcttgtatgccgagcaggacagcagtcattgcttcgtttccaccgtggaatgcaaagctgtcatggttttctttcactactgcgtggtgtccaactacttctggctgttcattgaaggcctatacctctttacactgctggtggagaccttcttccctgagaggagatatttctactggtataccatcattggctgggggacacctactgtgtgtgtaactgtgtgggctgtgctgaggctctactttgatgatgcgggatgctgggatatgaatgacagcacagctctgtggtgggtgatcaaaggccctgtagttggctctataatggttaactttgtgcttttcatcggcatcatcatcatccttgtgcagaagctgcagtccccagacatgggaggcaatgagtcgagcatctacttctgcgtgcagaaatgctactgcaagccacagcgggctcagcagcactcttgcaagatgtcagaactatccaccattactctacggctggcccgctccaccctgctgctcatcccactctttggaatccactacacagtatttgccttctctccagagaacgtcagcaagagggaaagacttgtgtttgagcttgggctgggctccttccagggctttgtggtggctgtactctactgcttcctgaatggggaggtacaggcagagattaagaggaaatggaggagctggaaggtgaaccgttacttcactatggacttcaagcaccggcatccatccctggccagcagtggagtgaacgggggcacccagctgtccatcctgagcaagagcagctcccagctccgcatgtccagcctcccggccgacaacttggccacctga

SEQ ID NO: 13 - protein, 63 aa, Mus musculus
CDATCQFRKAIEDCRKKAHHSDVLQTSVQTTATFTSMDTSQLPGSGVFKECMKEKAKEFKAGK